SEQUENCE LISTING (14 sequences)

SEQ ID NO: 1 - protein, 941 aa, Mus musculus
  Murine Rubicon (1)..(941)
MRPEGAGMDLGGGDGERLLEKSRREHWQLLGNLKTTVEGLVSANCPNVWSKYGGLERLCRDMQNILYHGLIHDQVCCRQADYWQFVKDIRWLSPHSALHVEKFISLHESDQSDTDSVSERAVAELWLQHSLQCHCLSAQLRPLLGDRQYIRKFYTETAFLLSDAHVTAMLQCLEAVEQNNPRLLAQIDASMFARKQESPLLVTKSQSLTALPGSTYTPPASYAQHSYFGSSSSLQSMPQSSHSSERRSTSFSLSGPSWQPQEDRECLSPAETQTTPAPLPSDSTLAQDSPLTAQEMSDSTLTSPLEASWVSSQNDSPSDVSEGPEYLAIGNPAPHGRTASCESHSSNGESSSSHLFSSSSSQKLESAASSLGDQEEGRQSQAGSVLRRSSFSEGQTAPVASGTKKSHIRSHSDTNIASRGAAEGGQYLCSGEGMFRRPSEGQSLISYLSEQDFGSCADLEKENAHFSISESLIAAIELMKCNMMSQCLEEEEVEEEDSDREIQELKQKIRLRRQQIRTKNLLPAYRETENGSFRVTSSSSQFSSRDSTQLSESGSAEDADDLEIQDADIRRSAVSNGKSSFSQNLSHCFLHSTSAEAVAMGLLKQFEGMQLPAASELEWLVPEHDAPQKLLPIPDSLPISPDDGQHADIYKLRIRVRGNLEWAPPRPQIIFNVHPAPTRKIAVAKQNYRCAGCGIRTDPDYIKRLRYCEYLGKYFCQCCHENAQMVVPSRILRKWDFSKYYVSNFSKDLLLKIWNDPLFNVQDINSALYRKVKLLNQVRLLRVQLYHMKNMFKTCRLAKELLDSFDVVPGHLTEDLHLYSLSDLTATKKGELGPRLAELTRAGAAHVERCMLCQAKGFICEFCQNEEDVIFPFELHKCRTCEECKACYHKTCFKSGRCPRCERLQARRELLAKQSLESYLSDYEEEPTEALALEATVLETT

SEQ ID NO: 2 - protein, 972 aa, Homo sapiens
MRPEGAGMELGGGEERLPEESRREHWQLLGNLKTTVEGLVSTNSPNVWSKYGGLERLCRDMQSILYHGLIRDQACRRQTDYWQFVKDIRWLSPHSALHVEKFISVHENDQSSADGASERAVAELWLQHSLQYHCLSAQLRPLLGDRQYIRKFYTDAAFLLSDAHVTAMLQCLEAVEQNNPRLLAQIDASMFARKHESPLLVTKSQSLTALPSSTYTPPNSYAQHSYFGSFSSLHQSVPNNGSERRSTSFPLSGPPRKPQESRGHVSPAEDQTIQAPPVSVSALARDSPLTPNEMSSSTLTSPIEASWVSSQNDSPGDASEGPEYLAIGNLDPRGRTASCQSHSSNAESSSSNLFSSSSSQKPDSAASSLGDQEGGGESQLSSVLRRSSFSEGQTLTVTSGAKKSHIRSHSDTSIASRGAPESCNDKAKLRGPLPYSGQSSEVSTPSSLYMEYEGGRYLCSGEGMFRRPSEGQSLISYLSEQDFGSCADLEKENAHFSISESLIAAIELMKCNMMSQCLEEEEVEEEDSDREIQELKQKIRLRRQQIRTKNLLPMYQEAEHGSFRVTSSSSQFSSRDSAQLSDSGSADEVDEFEIQDADIRRNTASSSKSFVSSQSFSHCFLHSTSAEAVAMGLLKQFEGMQLPAASELEWLVPEHDAPQKLLPIPDSLPISPDDGQHADIYKLRIRVRGNLEWAPPRPQIIFNVHPAPTRKIAVAKQNYRCAGCGIRTDPDYIKRLRYCEYLGKYFCQCCHENAQMAIPSRVLRKWDFSKYYVSNFSKDLLIKIWNDPLFNVQDINSALYRKVKLLNQVRLLRVQLCHMKNMFKTCRLAKELLDSFDTVPGHLTEDLHLYSLNDLTATRKGELGPRLAELTRAGATHVERCMLCQAKGFICEFCQNEDDIIFPFELHKCRTCEECKACYHKACFKSGSCPRCERLQARREALARQSLESYLSDYEEEPAEALALEAAVLEAT

SEQ ID NO: 3 - protein, 570 aa, Homo sapiens
  Human Nox2 (1)..(570)
MGNWAVNEGLSIFVILVWLGLNVFLFVWYYRVYDIPPKFFYTRKLLGSALALARAPAACLNFNCMLILLPVCRNLLSFLRGSSACCSTRVRRQLDRNLTFHKMVAWMIALHSAIHTIAHLFNVEWCVNARVNNSDPYSVALSELGDRQNESYLNFARKRIKNPEGGLYLAVTLLAGITGVVITLCLILIITSSTKTIRRSYFEVFWYTHHLFVIFFIGLAIHGAERIVRGQTAESLAVHNITVCEQKISEWGKIKECPIPQFAGNPPMTWKWIVGPMFLYLCERLVRFWRSQQKVVITKVVTHPFKTIELQMKKKGFKMEVGQYIFVKCPKVSKLEWHPFTLTSAPEEDFFSIHIRIVGDWTEGLFNACGCDKQEFQDAWKLPKIAVDGPFGTASEDVFSYEVVMLVGAGIGVTPFASILKSVWYKYCNNATNLKLKKIYFYWLCRDTHAFEWFADLLQLLESQMQERNNAGFLSYNIYLTGWDESQANHFAVHHDEEKDVITGLKQKTLYGRPNWDNEFKTIASQHPNTRIGVFLCGPEALAETLSKQSISNSESGPRGVHFIFNKENF

SEQ ID NO: 4 - protein, 570 aa, Mus musculus
  Murine Nox2 (1)..(570)
MGNWAVNEGLSIFVILVWLGLNVFLFINYYKVYDDGPKYNYTRKLLGSALALARAPAACLNFNCMLILLPVCRNLLSFLRGSSACCSTRIRRQLDRNLTFHKMVAWMIALHTAIHTIAHLFNVEWCVNARVGISDRYSIALSDIGDNENEEYLNFAREKIKNPEGGLYVAVTRLAGITGIVITLCLILIITSSTKTIRRSYFEVFWYTHHLFVIFFIGLAIHGAERIVRGQTAESLEEHNLDICADKIEEWGKIKECPVPKFAGNPPMTWKWIVGPMFLYLCERLVRFWRSQQKVVITKVVTHPFKTIELQMKKKGFKMEVGQYIFVKCPKVSKLEWHPFTLTSAPEEDFFSIHIRIVGDWTEGLFNACGCDKQEFQDAWKLPKIAVDGPFGTASEDVFSYEVVMLVGAGIGVTPFASILKSVWYKYCDNATSLKLKKIYFYWLCRDTHAFEWFADLLQLLETQMQERNNANFLSYNIYLTGWDESQANHFAVHHDEEKDVITGLKQKTLYGRPNWDNEFKTIASEHPNTTIGVFLCGPEALAETLSKQSISNSESGPRGVHFIFNKENF

SEQ ID NO: 5 - DNA, 21 bp, Artificial
  synthetic
  GAPDH-F-primer (1)..(21)
aggtcggtgtgaacggatttg

SEQ ID NO: 6 - DNA, 23 bp, Artificial
  synthetic
  GAPDH-R-primer (1)..(23)
tgtagaccatgtagttgaggtca

SEQ ID NO: 7 - DNA, 22 bp, Artificial
  synthetic
  IL-1beta-F-primer (1)..(22)
gcaactgttcctgaactcaact

SEQ ID NO: 8 - DNA, 21 bp, Artificial
  synthetic
  IL-1beta-R-primer (1)..(21)
atcttttggggtccgtcaact

SEQ ID NO: 9 - DNA, 21 bp, Artificial
  synthetic
  TNF-alpha-F-primer (1)..(21)
aggaggagtctgcgaagaaga

SEQ ID NO: 10 - DNA, 21 bp, Artificial
  synthetic
  TNF-alpha-R-primer (1)..(21)
ggcagtggaccatctaactcg

SEQ ID NO: 11 - DNA, 19 bp, Artificial
  synthetic
  TGF-beta-F-primer (1)..(19)
ctcccgtggcttctagtgc

SEQ ID NO: 12 - DNA, 22 bp, Artificial
  synthetic
  TGF-beta-R-primer (1)..(22)
gccttagtttggacaggatctg

SEQ ID NO: 13 - DNA, 21 bp, Artificial
  synthetic
  IL-10-F-primer (1)..(21)
gctcttactgactggcatgag

SEQ ID NO: 14 - DNA, 20 bp, Artificial
  synthetic
  IL-10-R-primer (1)..(20)
cgcagctctaggagcatgtg

(19) United States
(12) Patent Application Publication
(10) Pub. No.: US 2021/0283068 A1
(43) Pub. Date: Sep. 16, 2021

(54) TUMOR SUPPRESSION BY MODULATION OF NON-CANONICAL AUTOPHAGY (LAP) IN MYELOID CELLS

(71) Applicant: St. Jude Children's Research Hospital, Memphis, TN (US)

(72) Inventors: Douglas R. Green, Memphis, TN (US); Larissa Dias da Cunha, Memphis, TN (US)

(73) Assignee: St. Jude Children's Research Hospital, Memphis, TN (US)

(21) Appl. No.: 16/606,002

(22) PCT Filed: Apr. 18, 2018
(86) PCT No.: PCT/IB2018/052697

§ 371 Date: Oct. 17, 2019

Related U.S. Application Data
(60) Provisional application No. 62/487,195, filed on Apr. 19, 2017.

(51) Int. Cl. A61K 31/03; A61K 31/12; A61P 35/04

(52) U.S. Cl. CPC A61K 31/03; A61P 35/04; A61K 31/12

(57) ABSTRACT

Compositions and methods are provided for suppressing tumors by modulating the LAP pathway. Targeting components of the LAP pathway for specific drug design can be used as n immunotherapy strategy that modulates the tumor microenvironment. It is well established that infiltrating monocytes and macrophages play a pivotal role in shaping an immunosuppressive tumor microenvironment. By modulating LAP in the innate immune cells, the function of effector T cells can be manipulated toward an effective, cytotoxic immune response that can eliminate tumor cells. Thus, methods are provided for reducing the size or number of tumor cells and for treating cancer or other cell proliferative disorders. Further provided are methods for increasing the Th1 response or increasing IFNγ and/or TNFα expression in the tumor microenvironment by administering a LAP inhibitor.

### STATEMENT OF GOVERNMENT SUPPORT

This invention was made with government support under AI40646 awarded by the National Institutes of Health (NIH). The government has certain rights in the invention.

### FIELD OF THE INVENTION

The invention relates to the field of cancer biology and immunology. In particular, the invention relates to a method for modulating the LAP pathway in order to reduce tumor size or number. The methods and compositions can be used to treat cancer and other cell proliferative disorders.

### BACKGROUND OF THE INVENTION

Cancer is the second leading cause of death in the United States, exceeded only by heart disease. Despite recent advances in cancer diagnosis and treatment, surgery and radiotherapy may be curative if a cancer is found early, but current drug therapies for metastatic disease are mostly palliative and seldom offer a long-term cure. Even with new chemotherapies entering the market, the need continues for new drugs effective in monotherapy or in combination with existing agents as first line therapy, and as second and third line therapies in treatment of resistant tumors.

Cancer cells are by definition heterogeneous. For example, within a single tissue or cell type, multiple mutational “mechanisms” may lead to the development of cancer. As such, heterogeneity frequently exists between cancer cells taken from tumors of the same tissue and same type that have originated in different individuals. Frequently observed mutational “mechanisms” associated with some cancers may differ between one tissue type and another (e.g., frequently observed mutational “mechanisms” leading to colon cancer may differ from frequently observed “mechanisms” leading to leukemias). It is therefore often difficult to predict whether a particular cancer will respond to a particular chemotherapeutic agent (Cancer Medicine, 5th edition, Bast et al., B. C. Decker Inc., Hamilton, Ontario).

Components of cellular signal transduction pathways that regulate the growth and differentiation of normal cells can, when dysregulated, lead to the development of cellular proliferative disorders and cancer. Mutations in cellular signaling proteins may cause such proteins to become expressed or activated at inappropriate levels or at inappropriate times during the cell cycle, which in turn may lead to uncontrolled cellular growth or changes in cell-cell attachment properties. For example, dysregulation of receptor tyrosine kinases by mutation, gene rearrangement, gene amplification, and overexpression of both receptor and ligand has been implicated in the development and progression of human cancers. Macroautophagy (herein, autophagy) is a catabolic, cell survival mechanism activated during nutrient scarcity involving degradation and recycling of unnecessary or dysfunctional components. The proteins of autophagy machinery often interact with pathogens, such as Salmonella enterica, Listeria monocytogenes, Aspergillus fumigatus and Shigella flexneri, and function to quarantine and degrade invading organisms (xenophagy). LC3 (mammalian homologue of Atg8) is the most commonly monitored autophagy-related protein, and its lipidated form, LC3-II, is present on autophagosomes during canonical autophagy.

LC3-associated phagocytosis (LAP) is a process triggered following phagocytosis of particles that engage cell-surface receptors such as TLR1/2, TLR2/6, TLR4, TIM4 and FcR, resulting in recruitment of some, but not all, members of the autophagic machinery to stimulus-containing phagosomes, facilitating rapid phagosome maturation, degradation of engulfed pathogens, and modulation of immune responses. LAP and autophagy have been shown to be functionally and mechanistically distinct processes. Whereas the autophagosome is a double-membrane structure, the LAP-engaged phagosome (LAPosome) is composed of a single membrane. Autophagy requires the activity of the pre-initiation complex, but LAP does not. However, LAP requires some autophagic components, such as the Class III PI(3)K complex and elements of the ubiquitylation-like, protein conjugation systems (ATG5, ATG7).

There remain significant gaps in our ability to differentiate LAP from canonical autophagy, in terms of molecular mechanisms and specificity. The Class III PI(3)K-associated protein, Rubicon, has been identified as required for LAP, yet non-essential for autophagy. Rubicon facilitates VPS34 activity and sustained PtdIns(3)P presence on LAPosomes and stabilizes the NOX2 complex for reactive oxygen species (ROS) production, both of which are critical for progression of LAP.

Accordingly, there is a need in the art for new compounds and methods for modulating various genes and signaling pathways; and methods for treating proliferation disorders, including cancer. The present invention addresses these needs.

### SUMMARY OF THE INVENTION

Compositions and methods are provided for suppressing tumors by modulating the LAP pathway. Targeting components of the LAP pathway for specific drug design can be used as an immunotherapy strategy that modulates the tumor microenvironment. It is well established that infiltrating monocytes and macrophages play a pivotal role in shaping an immunosuppressive tumor microenvironment. By modulating LAP in the innate immune cells, the function of effector T cells can be manipulated toward an effective, cytotoxic immune response that can eliminate tumor cells. Thus, methods are provided for reducing the size or number of tumor cells and for treating cancer or other cell proliferative disorders. Further provided are methods for increasing the Th1 response or increasing IFNγ and/or TNFα expression in the tumor microenvironment by administering a LAP inhibitor.

### BRIEF DESCRIPTION OF THE FIGURES

FIG. 1 depicts that LAP-deficiency in myeloid cells is protective in a graft tumor model. (FIG. 1A.) Schematic representation of experimental design for FIG. 1B,C. Atg5^{floxflox} LysM Cre⁻ (Atg5^{f/f} Cre⁻) and Atg5^{floxflox} LysM Cre⁺ (Atg5^{f/f} Cre⁺) (FIG. 1B) or Fip200^{floxflox} LysM Cre⁻ (Fip200^{f/f} Cre⁻) and Fip200^{loxflox} LysM Cre⁺ (Fip200^{f/f} Cre⁺) (FIG. 1C) mice were injected subcutaneously in the rear flank with 2.5×10⁵ B16-F10 and tumor growth was estimated as indicated.

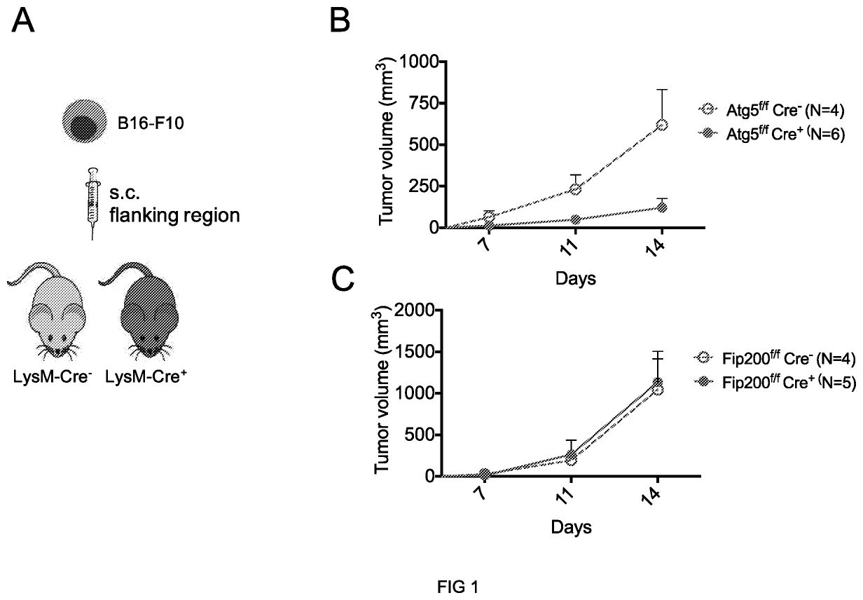

FIG. 2 shows that LAP-deficiency in myeloid cells is protective in a graft tumor model of melanoma and Lewis Lung carcinoma. (FIG. 2A) Schematic representation of experimental design for FIG. 2B-E. Atg5^{f/f} Cre⁻/Cre⁺ or Fip200^{f/f} Cre⁻/Cre⁺ mice were injected subcutaneously in the rear flank with 2.5×10⁵ B16-F10 (FIG. 2B-C) or 1.0×10⁵ LLC (FIG. 2D-E) cells and tumor growth was estimated.

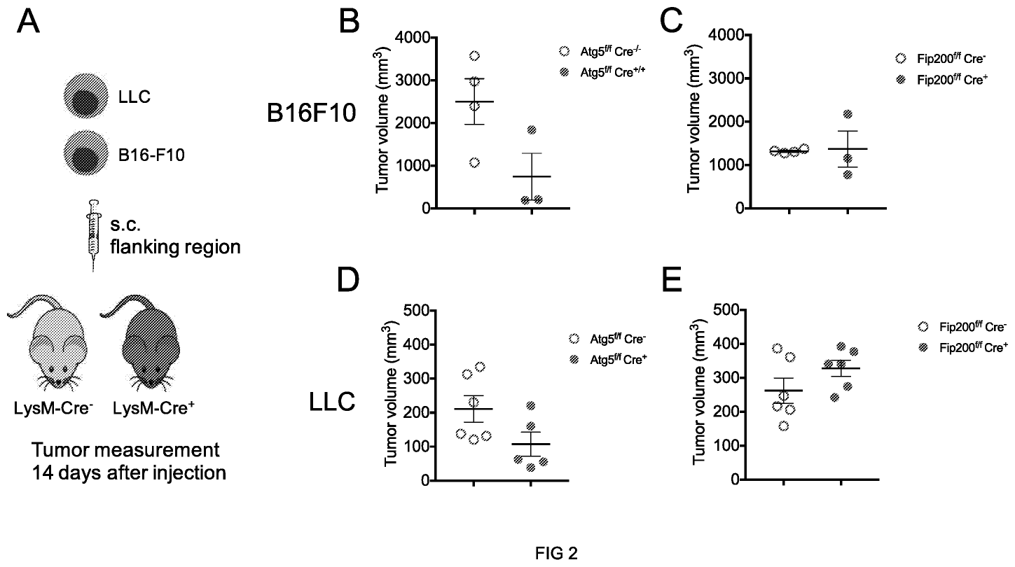

FIG. 3 shows a skew of CD4+ expression toward a Th1 response in the tumor microenvironment. (FIG. 3A) Schematic representation of experimental design for FIG. 3B-G. (FIG. 3B-G) Atg5^{f/f} Cre⁻/Cre⁺ or Fip200^{f/f} Cre⁻/Cre⁺ mice were injected subcutaneously in the rear flank with 2.5×10⁵ B16-F10 and tumor-infiltrating cells were purified and analyzed by flow cytometry 14 days post-injection. (FIG. 3B, E) Percentage of CD4⁺ lymphocytes within total CD45⁺ infiltrating cells. (FIG. 3C, F) Percentage of IFN-γ⁺ positive cells within CD4⁺ lymphocytes. (FIG. 3D,G) Geometric mean of fluorescence intensity (iMFI) of IFN-γ⁺ CD4⁺ cells.

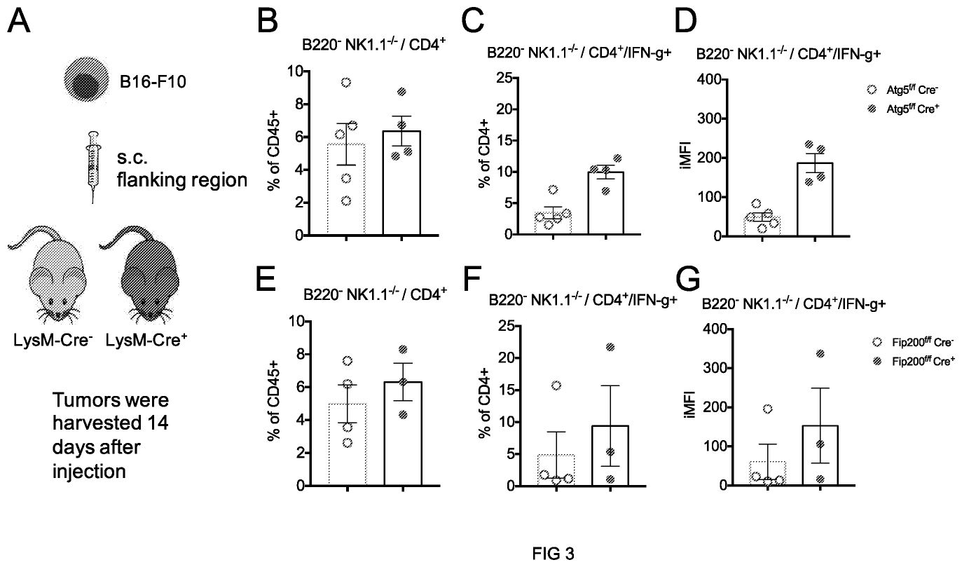

FIG. 4 demonstrates that LAP deficiency can produce more active cytotoxic T-lymphocytes in the tumor microenvironment. (FIG. 4A) Schematic representation of experimental design for FIG. 4B-G. (FIG. 4B-G) Atg5^{f/f} Cre⁻/Cre⁺ or Fip200^{f/f} Cre⁻/Cre⁺ mice were injected subcutaneously in the rear flank with 2.5×10⁵ B16-F10 and tumor-infiltrating cells were purified and analyzed by flow cytometry 14 days post-injection. (FIG. 4B, E) Percentage of CD8⁺ lymphocytes within total CD45⁺ infiltrating cells. (FIG. 4C, F) Percentage of IFN-γ⁺ positive cells within the population of CD8⁺ lymphocytes. (FIG. 4D,G) Geometric mean of fluorescence intensity (iMFI) of IFN-γ⁺ CD8⁺ cells.

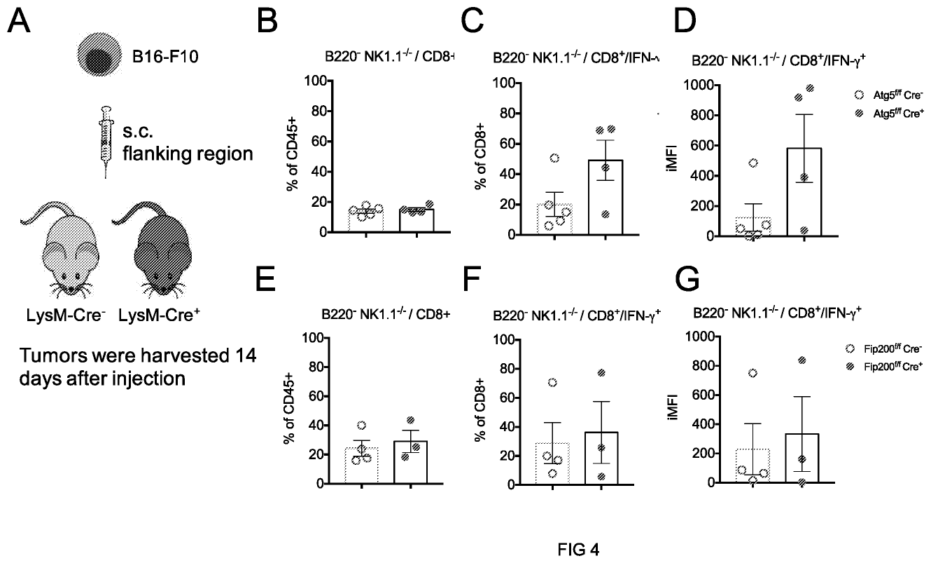

FIG. 5 depicts that LAP deficiency can skew macrophage polarization toward M1 macrophages. (FIG. 5A-H) Atg5^{f/f} Cre⁻/Cre⁺ or Fip200^{f/f} Cre⁻/Cre⁺ mice were injected subcutaneously in the rear flank with 2.5×10⁵ B16-F10 (FIG. 5A, B, E, F) or 1.0×10⁵ LLC (FIG. 5C, D, G, H) cells and tumor-infiltrating cells were purified and analyzed by flow cytometry 14 days post-injection. (FIG. 5A-D) Geometric mean of fluorescence intensity (iMFI) of CD11c⁺ (M1 marker) of Ly-6G⁻/F4/80⁺ cells within myeloid-derived infiltrating cells. (FIG. 5E-H) Geometric mean of fluorescence intensity (iMFI) of CD206⁺ (M2 marker) of Ly-6G⁻/F4/80⁺ cells within myeloid-derived infiltrating cells.

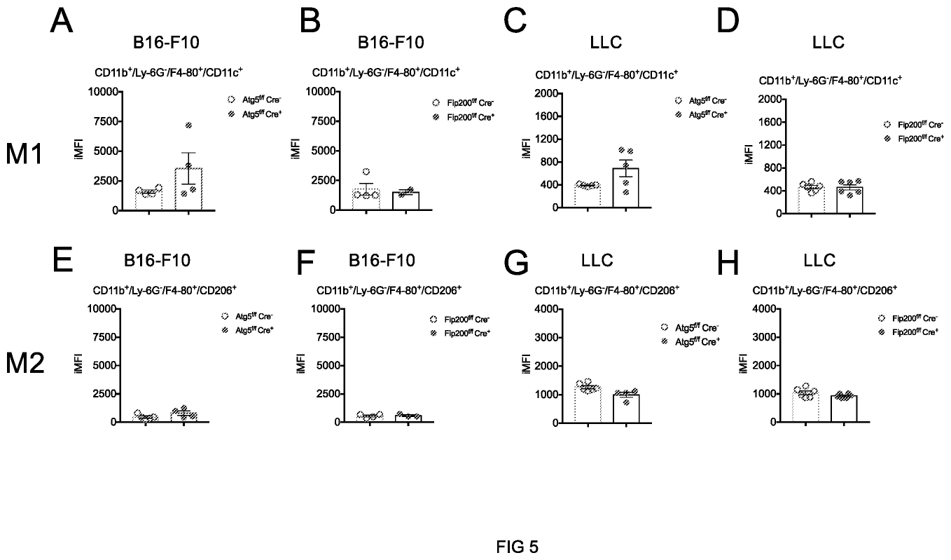

FIG. 6 also depicts that LAP deficiency can skew macrophage polarization toward M1 macrophages in a melanoma model. (FIG. 6A) Schematic representation of experimental design for FIG. 6B-E. (FIG. 6B-E) Atg5^{f/f} or Fip200^{f/f} Cre⁻ (open bars) and Cre⁺ (filled bars) mice were injected subcutaneously in the rear flank with 1.0×10⁵ LLC cells and tumor-infiltrating cells were purified and 14 days post-injection. Expression of target genes was evaluated by qRT-PCR.

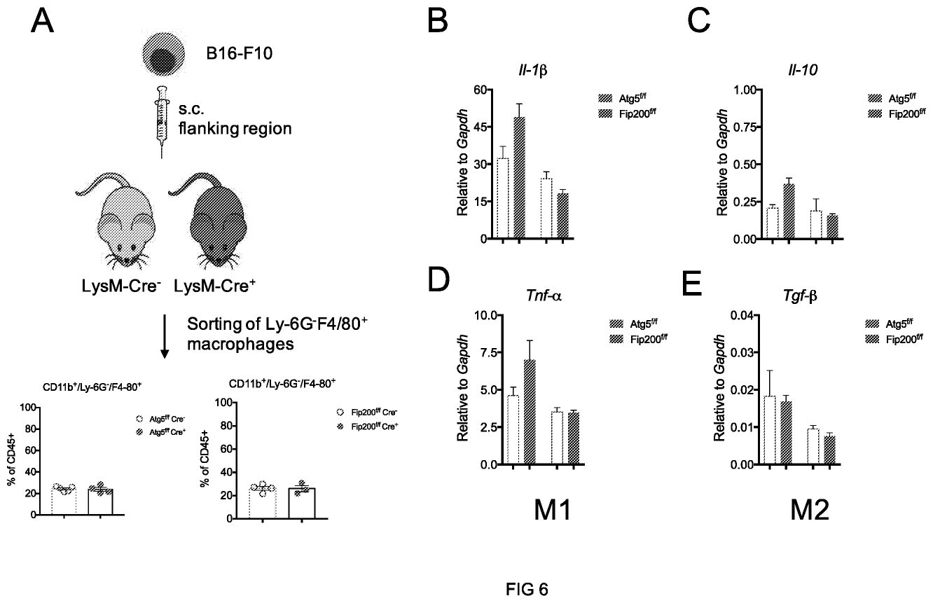

FIG. 7 demonstrates that the tumor-suppressive effect of LAP-impairment in myeloid cells requires CD4⁺ and CD8⁺ lymphocytes. (FIG. 7A) Schematic representation of experimental design for FIG. 7B. (FIG. 7B) Atg5^{f/f} Cre⁻/Cre⁺ mice were injected subcutaneously in the rear flank with 1.0×10⁵ LLC cells. The day before and at days 5 and 9 post-injection, mice were treated intraperitoneally with 150 mg of depleting antibodies against CD4 and CD8 or with isotype control, as indicated. Tumor growth was estimated with caliper.

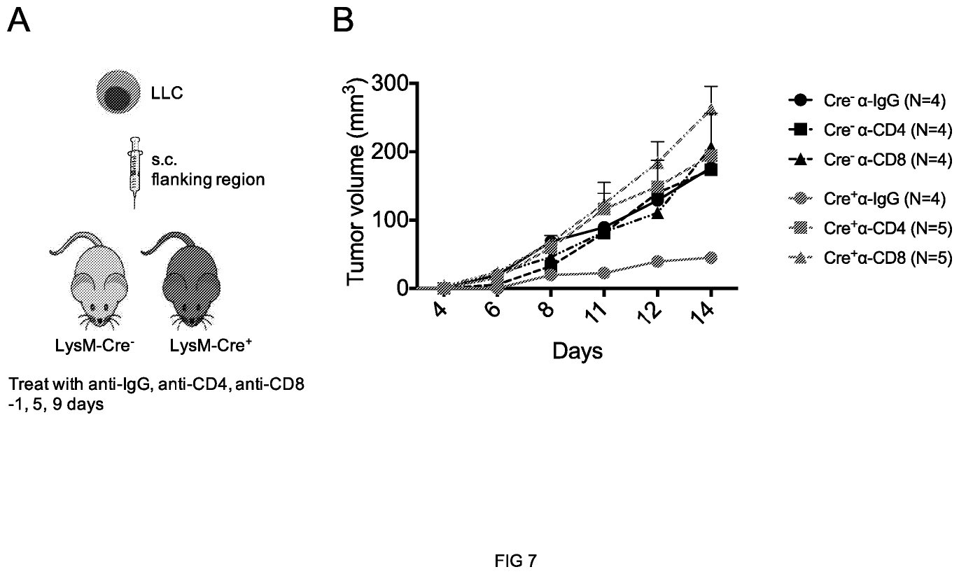

FIG. 8 shows that LAP-deficiency in myeloid cells is protective in a model of tumor metastasis. (FIG. 8A) Schematic representation of experimental design for FIG. 8B-E. (FIG. 8B-E) 1.0×10⁵ B16-F10 cells were intravenously injected and the number of micrometastatic spots in the lungs were counted 21 days post-injection.

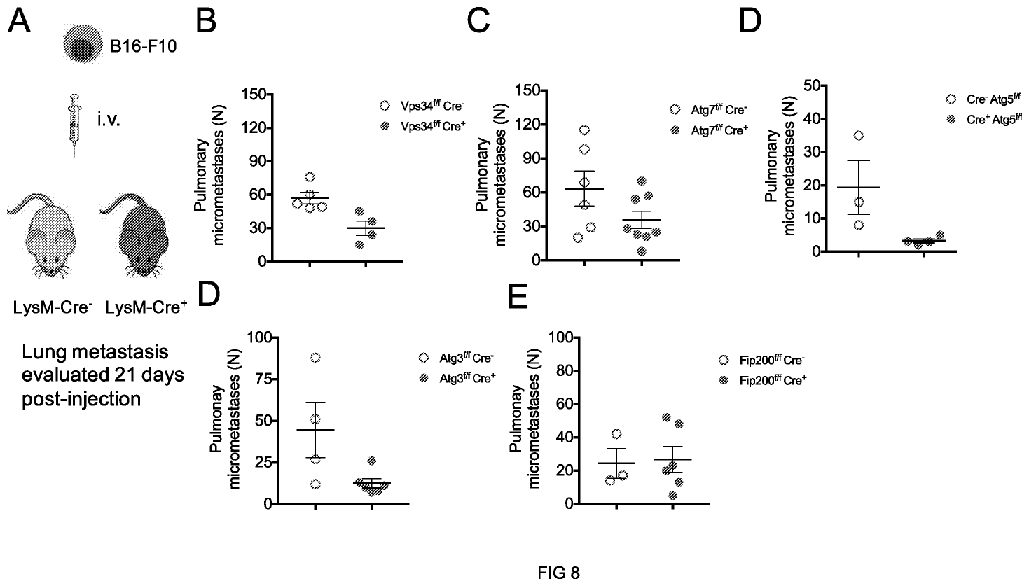

FIG. 9 depicts that inhibition of NOX2 and scavenging of reactive oxygen species (ROS) abrogates LAP activity in vitro. (FIG. 9A-FIG. 9B) Evaluation of ROS scavenging in non-treated cells (NT) and cells treated with apocynin (NOX2 inhibitor) or DPI (ROS scavenger), as measured by flow cytometry using the ROS probe H2DCFDA (DCF, Molecular probes) at 2.5 μM. RAW cells were non-stimulated (NS) or stimulated with zymozan-Alexa594 (ZYM) for 1 h in the presence of the compounds described above (treatment started 30 min prior to stimulation with zymozan). (FIG. 9C) Quantification of FIG. 9.A-FIG. 9B, in MFI. (FIG. 9D) Evaluation of LAP, as assessed by LC3-II lipidation upon stimuli. RAW cells expressing Venus-LC3 were stimulated with ZYM for 1 h, treated with digitonin and accumulation of lipidated LC3-II in permeabilized cells was measured by flow cytometry. (FIG. 9E) Quantification of FIG. 9D, in MFI. (FIG. 9F) Assessment of zymozan engulfment by treated cells, measured by flow cytometry.

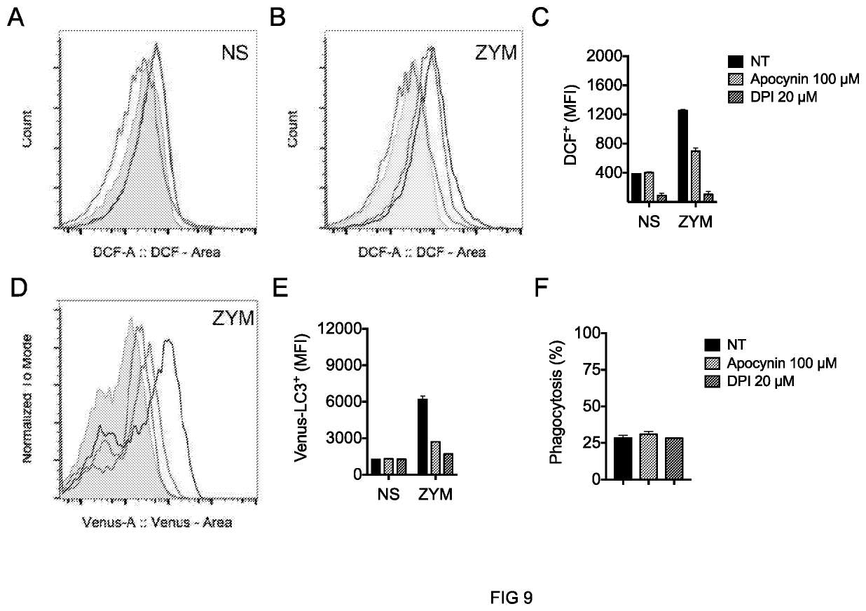

### DETAILED DESCRIPTION OF THE INVENTION

The present inventions now will be described more fully hereinafter with reference to the accompanying drawings, in which some, but not all embodiments of the inventions are shown. Indeed, these inventions may be embodied in many different forms and should not be construed as limited to the embodiments set forth herein; rather, these embodiments are provided so that this disclosure will satisfy applicable legal requirements. Like numbers refer to like elements throughout.

Many modifications and other embodiments of the inventions set forth herein will come to mind to one skilled in the art to which these inventions pertain having the benefit of the teachings presented in the foregoing descriptions and the associated drawings. Therefore, it is to be understood that the inventions are not to be limited to the specific embodiments disclosed and that modifications and other embodiments are intended to be included within the scope of the appended claims. Although specific terms are employed herein, they are used in a generic and descriptive sense only and not for purposes of limitation.

### 1. Overview

Compositions and methods are provided herein for the treatment of cancer by administering to a patient having cancer a LC3-associated phagocytosis (LAP) inhibitor. LAP is a process in which some, but not all components of the autophagy machinery conjugate myosin associated light chain-3 (LC3) to phosphatidylethanolamine directly on the phagosome membrane, and the lipidated LC3 (LC3-II) then functions to facilitate lysosomal fusion and cargo destruction (e.g., LAP activity). Both LAP and canonical autophagy require ATG7, ATG3, ATG5, ATG12, and ATG16L for the process of LC3 lipidation. However, unlike canonical autophagy, LAP proceeds independently of the autophagic pre-initiation complex containing ULK1 and FIP200, and utilizes a distinct Beclin 1-VPS34 complex lacking ATG14. In contrast, LAP, but not canonical autophagy, requires NADPH oxidase-2 (NOX2), and Rubicon. These requirements for LAP and canonical autophagy can therefore distinguish between these two processes (See, Table 1). As used herein, the term “LAP-related” refers to any nucleic acid, protein, cytokine, or any other molecule that participates in the LAP pathway. LAP-related molecules include, but are not limited to Beclin1, BPS34, UVRAG, ATG7, ATG3, ATG5, ATG12, ATG16L, ATG3, ATG4, LC3 family LC3A, LC3B, GATE16, GABARAP), Rubicon, and NOX2. See, Table 1 for a description of selected LAP-related molecules and their associated function.

TABLE 1

| | | |
|---|---|---|
| Component | Function/Complex | Reference |

Components required for canonical autophagy and LAP

| | | |
|---|---|---|
| Beclin1 | Class III PI3K complex | 1, 2, 4, 6 |
| VPS34 | Class III PI3K complex | 1, 2, 4, 6 |
| UVRAG | Class III PI3K complex, vesicle sorting | 6 |
| ATG5 | ATG5-12-16L complex formation, complex | 1, 2, 4, 6 |
| | functions as E3 for LC3-II generation | |
| ATG12 | ATG5-12-16L complex formation, complex | 6 |
| | functions as E3 for LC3-II generation | |
| ATG16L | ATG5-12-16L complex formation, complex | 6 |
| | functions as E3 for LC3-II generation | |
| ATG7 | ATG5-12-16L complex and LC3-PE formation | 1-6 |
| | (functions as E1) | |
| ATG3 | LC3-PE formation (function as E2) | 6 |
| ATG4 | LC3 processing | 6 |
| LC3 family (LC3A, | Maturation and fusion to lysosomal | |
| LC3B, GATE16, | compartments | 6 |
| GABARAP) | | |

Components required for canonical autophagy only

| | | |
|---|---|---|
| ULK1 | Pre-initiation complex | 2-6 |
| FIP200 | Pre-initiation complex | 4-6 |
| ATG13 | Pre-initiation complex | 4-6 |
| Ambra1 | Class III PI3K complex | 6 |
| WIPI2 | Recruitment of ATG5-12-16L | 6 |
| ATG14 | Class III PI3K complex | 6 |

Components required for LAP only

| | | |
|---|---|---|
| Rubicon | Localization and activity of Class III PI3K | 6 |
| | complex, stabilization of NOX2 complex | |
| NOX2 | NADPH oxidase, ROS production, recruitment of ATG5- | 6 |
| | 12-16L and LC3 conjugation systems | |

1. Sanjuan, M. A. et al. Toll-like receptor signalling in macrophages links the autophagy pathway to phagocytosis. Nature 450, 1253-1257 (2007).
2. Martinez, J. et al. Microtubule-associated protein 1 light chain 3 alpha (LC3)-associated phagocytosis is required for the efficient clearance of dead cells. Proceedings of the National Academy of Sciences of the United States of America 108, 17396-17401, doi:10.1073/pnas.1113421108 (2011).
3. Florey, O., Kim, S. E., Sandoval, C. P., Haynes, C. M. & Overholtzer, M. Autophagy machinery mediates macroendocytic processing and entotic cell death by targeting single membranes. Nat Cell Biol 13, 1335-1343, doi:10.1038/ncb2363 (2011).
4. Henault, J. et al. Noncanonical autophagy is required for type I interferon secretion in response to DNA-immune complexes. Immunity 37, 986-997, doi:10.1016/j.immuni.2012.09.014 (2012).
5. Kim, J. Y. et al. Noncanonical autophagy promotes the visual cycle. Cell 154, 365-376, doi:10.1016/j.cell.2013.06.012 (2013).
6. Martinez, J. et al. Molecular characterization of LC3-associated phagocytosis (LAP) reveals distinct roles for Rubicon, NOX2, and autophagy proteins. Nature cell biology, 17, 893-906.

Accordingly, the term “LAP inhibitor” refers to any molecule capable of reducing the expression or activity of a LAP-related molecule, or capable of reducing LAP activity in a cell. Likewise, a “LAP inhibitor” can be any molecule that reduces the expression or activity of a LAP-related molecule, or that reduces LAP activity in a cell. A LAP inhibitor could be a nucleic acid molecule, a protein, a chemical compound, a small molecule, or any composition that reduces the expression or activity of a LAP-related molecule, or reduces the LAP activity in a cell. LAP activity can be determined by measuring dead cell clearance or by the methods disclosed herein in the Examples. One method to monitor LAP or LAP activity is to use Western blot analysis to identify key components such as Rubicon and LC3-II. Further, as disclosed elsewhere herein, LAP activity can be measured using immunofluorescence to identify LC3 associated with phagosomes, or flow cytometry. Any method known in the art can be used for measuring LAP activity, including those described in Martinez et al. (2015) Nature Cell Biology 17: 893-906, herein incorporated by reference in the entirety. In specific embodiments, administration of an effective amount of a LAP-inhibitor can reduce the size or number of tumors or can reduce the spread, severity, or progression of the cancer.

### 2. Methods of Treatment

“Treatment” or “treating” as used herein refers to curing, healing, alleviating, relieving, altering, remedying, ameliorating, improving, or affecting the condition or the symptoms of a cancer or cell proliferative disorder in a subject by reducing the expression or activity of a LAP-related molecule or by reducing the LAP activity of a cell. As used herein the term “symptom” refers to an indication of disease, illness, injury, or that something is not right in the body.

Symptoms are felt or noticed by the individual experiencing the symptom, but may not easily be noticed by others. Others are defined as non-health-care professionals. Cancer is a group of diseases that may cause almost any sign or symptom. The signs and symptoms will depend on where the cancer is, the size of the cancer, and how much it affects the nearby organs or structures. If a cancer spreads (metastasizes), then symptoms may appear in different parts of the body. As a cancer grows, it begins to push on nearby organs, blood vessels, and nerves. This pressure creates some of the signs and symptoms of cancer. If the cancer is in a critical area, such as certain parts of the brain, even the smallest tumor can cause early symptoms.

Sometimes cancers start in places where it does not cause any symptoms until the cancer has grown quite large. Pancreas cancers, for example, do not usually grow large enough to be felt from the outside of the body. Some pancreatic cancers do not cause symptoms until they begin to grow around nearby nerves (this causes a backache). Others grow around the bile duct, which blocks the flow of bile and leads to a yellowing of the skin known as jaundice. By the time a pancreatic cancer causes these signs or symptoms, it has usually reached an advanced stage. Cancer presents several general signs or symptoms that occur when a variety of subtypes of cancer cells are present. Most people with cancer will lose weight at some time with their disease. An unexplained (unintentional) weight loss of 10 pounds or more may be the first sign of cancer, particularly cancers of the pancreas, stomach, esophagus, or lung.

Fever is very common with cancer, but is more often seen in advanced disease. Almost all patients with cancer will have fever at some time, especially if the cancer or its treatment affects the immune system and makes it harder for the body to fight infection. Less often, fever may be an early sign of cancer, such as with leukemia or lymphoma. Fatigue may be an important symptom as cancer progresses. It may happen early, though, in cancers such as with leukemia, or if the cancer is causing an ongoing loss of blood, as in some colon or stomach cancers.

Pain may be an early symptom with some cancers such as bone cancers or testicular cancer. But most often pain is a symptom of advanced disease. Along with cancers of the skin, some internal cancers can cause skin signs that can be seen. These changes include the skin looking darker (hyperpigmentation), yellow (jaundice), or red (erythema); itching; or excessive hair growth. In some cases, cancer subtypes present specific signs or symptoms. Changes in bowel habits or bladder function could indicate cancer. Long-term constipation, diarrhea, or a change in the size of the stool may be a sign of colon cancer. Pain with urination, blood in the urine, or a change in bladder function (such as more frequent or less frequent urination) could be related to bladder or prostate cancer.

Changes in skin condition or appearance of a new skin condition could be a symptom of cancer. Skin cancers may bleed and look like sores that do not heal. A long-lasting sore in the mouth could be an oral cancer, especially in patients who smoke, chew tobacco, or frequently drink alcohol. Sores on the penis or vagina may either be signs of infection or an early cancer. Unusual bleeding or discharge could indicate cancer. Unusual bleeding can happen in either early or advanced cancer. Blood in the sputum (phlegm) may be a sign of lung cancer. Blood in the stool (or a dark or black stool) could be a sign of colon or rectal cancer. Cancer of the cervix or the endometrium (lining of the uterus) can cause vaginal bleeding. Blood in the urine may be a sign of bladder or kidney cancer. A bloody discharge from the nipple may be a sign of breast cancer.

A thickening or lump in the breast or in other parts of the body could indicate the presence of a cancer. Many cancers can be felt through the skin, mostly in the breast, testicle, lymph nodes (glands), and the soft tissues of the body. A lump or thickening may be an early or late sign of cancer. Any lump or thickening could be indicative of cancer, especially if the formation is new or has grown in size. Indigestion or trouble swallowing could be symptomatic of cancer. While these symptoms commonly have other causes, indigestion or swallowing problems may be a sign of cancer of the esophagus, stomach, or pharynx (throat).

Recent changes in a wart or mole could be indicative of cancer. Any wart, mole, or freckle that changes in color, size, or shape, or loses its definite borders indicates the potential development of cancer. For example, the skin lesion may be a melanoma. A persistent cough or hoarseness could be indicative of cancer. A cough that does not go away may be a sign of lung cancer. Hoarseness can be a sign of cancer of the larynx (voice box) or thyroid.

While the signs and symptoms listed above are the more common ones seen with cancer, there are many others that are less common and are not listed here. However, all art-recognized signs and symptoms of cancer are contemplated and encompassed by the instant invention.

In specific embodiments, treatment or treating encompasses a reduction in the size of a tumor disclosed herein. Tumor size can be determined using a variety of methods known in the art, such as, for example, by measuring the dimensions of tumor(s) upon removal from the subject, e.g., using calipers, or while in the body using imaging techniques, e.g., ultrasound, computed tomography (CT) or magnetic resonance imaging (MRI) scans. Tumor size can be determined, for example, by determining tumor weight or tumor volume. As used herein, a reduction of tumor size refers to a rejection of the tumor diameter or tumor volume. The decrease in size can be, for example, a decrease of tumor diameter of 0.01 mm, 0.05 mm, 0.10 mm, 0.12 mm, 0.14 mm, 0.16 mm, 0.18 mm, 0.20 mm, 0.25 mm, 0.30 mm, 0.35 mm, 0.40 mm, 0.45 mm, 0.50 mm, 0.6 mm, 0.7 mm, 0.8 mm, 0.9 mm, 1.0 mm, 1.1 mm, 1.2 mm, 1.3 mm, 1.4 mm, 1.5 mm, 1.75 mm, 2.0 mm, 3.0 mm, 4.0 mm, 5.0 mm, 6.0 mm, 7.0 mm, 8.0 mm, 9.0 mm, 10.0 mm or more. The decrease in size can be a decrease in tumor volume of 10 mm³, 20 mm³, 30 mm³, 40 mm³, 50 mm³, 75 mm³, 100 mm³, 150 mm³, 200 mm³, 250 mm³, 300 mm³, 350 mm³, 400 mm³, 500 mm³, 600 mm³, 700 mm³, 800 mm³, 900 mm³, 1000 mm³ or more. In specific embodiments, such decreases or reductions in tumor size can be, for example, at least a 5%, 10%, 20%, 30%, 40%, 50%, 60%, 70%, 80%, 90%, 95%, 96%, 97%, 98%, 99%, 100%, 5-10%, 10-20%, 10-30%, 10-40%, 20-30%, 20-40%, 30-40%, 30-50%, 40-50%, 40-60%, 50-60%, 50-70%, 60-70%, 60-80%, 70-80%, 70-90%, 80-90%, 80-100%, 90-100%, or 95-100% reduction in tumor size. In specific embodiments, treatment or treating encompasses a reduction in the number of tumors in a subject. The decrease in tumor number can be a decrease of at least about 1, 2, 3, 4, 5, 6, 7, 8, 9, 10, 11, 12, 13, 14, 15, 16, 17, 18, 19, 20, 25, 30, 35, 40, 45, 50, 60, 70, 80, 90, or more tumors in a subject.

In some embodiments, treatment or treating encompasses a reduction in the spread or the progression of a cancer. The spread or progression of cancer can be reduced by at least about 5%, 10%, 20%, 30%, 40%, 50%, 60%, 70%, 80%, 90%, 95%, 96%, 97%, 98%, 99%, 100%, 5-10%, 10-20%, 10-30%, 10-40%, 20-30%, 20-40%, 30-40%, 30-50%, 40-50%, 40-60%, 50-60%, 50-70%, 60-70%, 60-80%, 70-80%, 70-90%, 80-90%, 80-100%, 90-100%, or 95-100% when compared to a proper control. The spread or progression of cancer can be determined by measuring the tumor size, tumor number, tumor location, or any other method known in the art for measuring spread or progression of cancer.

Treating cancer can result in a decrease in number of metastatic lesions in other tissues or organs distant from the primary tumor site. Preferably, after treatment, the number of metastatic lesions is reduced by 5%, 10%, 20%, 30%, 40%, 50%, 60%, 70%, 80%, 90%, 95%, 96%, 97%, 98%, 99%, 100%, 5-10%, 10-20%, 10-30%, 10-40%, 20-30%, 20-40%, 30-40%, 30-50%, 40-50%, 40-60%, 50-60%, 50-70%, 60-70%, 60-80%, 70-80%, 70-90%, 80-90%, 80-100%, 90-100%, or 95-100% compared to the number of metastatic lesions prior to administration of a LAP inhibitor. The number of metastatic lesions may be measured by any reproducible means of measurement. The number of metastatic lesions may be measured by counting metastatic lesions visible to the naked eye or at a specified magnification.

Treating cancer can result in an increase in average survival time of a population of treated subjects in comparison to a population of untreated subjects. In some embodiments, the average survival time is increased by more than 30 days; more preferably, by more than 60 days; more preferably, by more than 90 days; and most preferably, by more than 120 days. An increase in average survival time of a population may be measured by any reproducible means. An increase in average survival time of a population may be measured, for example, by calculating for a population the average length of survival following initiation of treatment with an active compound. An increase in average survival time of a population may also be measured, for example, by calculating for a population the average length of survival following completion of a first round of treatment with an active compound.

Treating cancer can result in a decrease in the mortality rate of a population of treated subjects in comparison to a population receiving carrier alone. Treating cancer can result in a decrease in the mortality rate of a population of treated subjects in comparison to an untreated population. Treating cancer can result in a decrease in the mortality rate of a population of treated subjects in comparison to a population receiving monotherapy with a drug that is not a LAP inhibitor. Preferably, the mortality rate is decreased by more than 2%, 5%, 10%, 20%, 30%, 40%, 50%, 60%, 70%, 80%, 90%, 95%, 96%, 97%, 98%, 99%, 100%, 5-10%, 10-20%, 10-30%, 10-40%, 20-30%, 20-40%, 30-40%, 30-50%, 40-50%, 40-60%, 50-60%, 50-70%, 60-70%, 60-80%, 70-80%, 70-90%, 80-90%, 80-100%, 90-100%, or 95-100%. A decrease in the mortality rate of a population of treated subjects may be measured by any reproducible means. A decrease in the mortality rate of a population may be measured, for example, by calculating for a population the average number of disease-related deaths per unit time following initiation of treatment with a LAP inhibitor. A decrease in the mortality rate of a population may also be measured, for example, by calculating for a population the average number of disease-related deaths per unit time following completion of a first round of treatment with an active compound.

Treating cancer can result in a decrease in tumor growth rate. Preferably, after treatment, tumor growth rate is reduced by at least 5%, 10%, 20%, 30%, 40%, 50%, 60%, 70%, 80%, 90%, 95%, 96%, 97%, 98%, 99%, 100%, 5-10%, 10-20%, 10-30%, 10-40%, 20-30%, 20-40%, 30-40%, 30-50%, 40-50%, 40-60%, 50-60%, 50-70%, 60-70%, 60-80%, 70-80%, 70-90%, 80-90%, 80-100%, 90-100%, or 95-100% relative to the rate prior to administration of the LAP inhibitor. Tumor growth rate may be measured by any reproducible means of measurement. Tumor growth rate can be measured according to a change in tumor diameter per unit time.

Treating cancer can result in a decrease in tumor regrowth. Preferably, after treatment, tumor regrowth is less than 5%, 10%, 20%, 30%, 40%, 50%, 60%, 70%, 80%, 90%, 95%, 96%, 97%, 98%, 99%, 100%, 5-10%, 10-20%, 10-30%, 10-40%, 20-30%, 20-40%, 30-40%, 30-50%, 40-50%, 40-60%, 50-60%, 50-70%, 60-70%, 60-80%, 70-80%, 70-90%, 80-90%, 80-100%, 90-100%, or 95-100%. Tumor regrowth may be measured by any reproducible means of measurement. Tumor regrowth is measured, for example, by measuring an increase in the diameter of a tumor after a prior tumor shrinkage that followed treatment. A decrease in tumor regrowth is indicated by failure of tumors to reoccur after treatment has stopped.

Treating or preventing a cell proliferative disorder can result in a reduction in the proportion of proliferating cells. Preferably, after treatment, the proportion of proliferating cells is reduced by at least 5%; more preferably, by at least 10%; more preferably, by at least 20%; more preferably, by at least 30%; more preferably, by at least 40%; more preferably, by at least 50%; even more preferably, by at least 50%; and most preferably, by at least 75%. The proportion of proliferating cells may be measured by any reproducible means of measurement. Preferably, the proportion of proliferating cells is measured, for example, by quantifying the number of dividing cells relative to the number of nondividing cells in a tissue sample. The proportion of proliferating cells can be equivalent to the mitotic index.

Treating or preventing a cell proliferative disorder can result in a decrease in the number or proportion of cells having an abnormal appearance or morphology. Preferably, after treatment, the number of cells having an abnormal morphology is reduced by at least 5% %, 10%, 20%, 30%, 40%, 50%, 60%, 70%, 80%, 90%, 95%, 96%, 97%, 98%, 99%, 100%, 5-10%, 10-20%, 10-30%, 10-40%, 20-30%, 20-40%, 30-40%, 30-50%, 40-50%, 40-60%, 50-60%, 50-70%, 60-70%, 60-80%, 70-80%, 70-90%, 80-90%, 80-100%, 90-100%, or 95-100% relative to the same measurement prior to treatment with a LAP inhibitor. An abnormal cellular appearance or morphology may be measured by any reproducible means of measurement. An abnormal cellular morphology can be measured by microscopy, e.g., using an inverted tissue culture microscope. An abnormal cellular morphology can take the form of nuclear pleiomorphism.

A cancer that is to be treated can be evaluated by DNA cytometry, flow cytometry, or image cytometry. A cancer that is to be treated can be typed as having 10%, 20%, 30%, 40%, 50%, 60%, 70%, 80%, or 90% of cells in the synthesis stage of cell division (e.g., in S phase of cell division). A cancer that is to be treated can be typed as having a low S-phase fraction or a high S-phase fraction.

In some embodiments, the subject is a LAP-deficient subject having reduced expression of a LAP-related molecule. As used herein, the term “reduced” refers to any reduction in the expression or activity of a LAP-related molecule when compared to the corresponding expression or activity of the same LAP-related molecule in a control cell. Such a reduction may be up to 5%, 10%, 20%, 30%, 40%, 50%, 60%, 70%, 80%, 90%, 95%, or up to 100%. Accordingly, the term “reduced” encompasses both a partial knockdown and a complete knockdown of the activity of a LAP-related molecule.

As described herein, a LAP-inhibitor can be administered in an effective amount in order to treat the cancer or cell proliferative disorder in the subject. In certain embodiments, an “effective amount” or a “therapeutically effective amount” of a LAP inhibitor can be sufficient to achieve a desired clinical result, including but not limited to, for example, ameliorating disease, stabilizing a subject, preventing or delaying the development of, or progression of, a proliferative disease, disorder, or condition in a subject. In specific embodiments, an effective amount is any amount sufficient to treat cancer or a cell proliferative disorder as described herein. For example, an effective amount is any amount of a LAP-inhibitor sufficient to reduce the tumor size, tumor number, reduce tumor spread, or reduce the progression of a cancer or cell-proliferative disorder. An effective amount of therapy can be determined based on one administration or repeated administration. Methods of detection and measurement of the indicators above are known to those of skill in the art. Such methods include, but are not limited to measuring reduction in tumor burden, reduction of tumor size, reduction of tumor volume, reduction in proliferation of secondary tumors, decreased solid tumor vascularization, expression of genes in tumor tissue, presence of biomarkers, lymph node involvement, histologic grade, and nuclear grade. “Positive therapeutic response” refers to, for example, improving the condition of at least one of the symptoms of a cancer, decreasing tumor size or tumor number, and/or reducing the progression of the cancer or cell proliferation disorder.

The specific therapeutically effective dose level for any particular subject will depend upon a variety of factors including the disorder being treated and the severity of the disorder; activity of the specific LAP-inhibitor employed; the specific composition employed; the age, body weight, general health, sex and diet of the patient; the time of administration; the route of administration; the rate of excretion of the composition employed; the duration of the treatment; drugs used in combination or coincidental with the specific compound employed; and like factors well known in the medical arts (see e.g., Koda-Kimble et al., (2004), Applied Therapeutics: The Clinical Use of Drugs, Lippincott Williams & Wilkins, ISBN 0781748453; Winter, (2003), Basic Clinical Pharmacokinetics, 4.sup.th ed., Lippincott Williams & Wilkins, ISBN 0781741475; Sharqel, (2004), Applied Biopharmaceutics & Pharmacokinetics, McGraw-Hill/Appleton & Lange, ISBN 0071375503). For example, it is well within the skill of the art to start doses of agents at levels lower than those required to achieve the desired therapeutic effect and to gradually increase the dosage until the desired effect can be achieved. If desired, the effective daily dose may be divided into multiple doses for purposes of administration. Consequently, single dose compositions may contain such amounts or submultiples thereof to make up the daily dose. It will be understood, however, that the total daily usage of the compounds and compositions of the present disclosure will be decided by an attending physician within the scope of sound medical judgment.

Administration of compositions described herein can occur as a single event, a periodic event, or over a time course of treatment. For example, agents can be administered daily, weekly, bi-weekly, or monthly. As another example, agents can be administered in multiple treatment sessions, such as 2 weeks on, 2 weeks off, and then repeated twice; or every 3rd day for 3 weeks. A first composition including a ligand coupled to molecule or substrate and a second composition including a receptor coupled to a radioisotope can have the same or different administration protocols. One of ordinary skill will understand these regimes to be exemplary and could design other suitable periodic regimes. For treatment of acute conditions, the time course of treatment will usually be at least several days. Certain conditions could extend treatment from several days to several weeks. For example, treatment could extend over one week, two weeks, or three weeks. For more chronic conditions, treatment could extend from several weeks to several months or even a year or more.

A. Treatment of Cancer

Methods and compositions are provided herein for treating cancer in a subject having cancer. As used herein, “cancer” refers to any cell-proliferative disorder in which unregulated or abnormal growth, or both, of cells can lead to the development of an unwanted condition or disease. Exemplary cell proliferative disorders of the invention encompass a variety of conditions wherein cell division is deregulated. Exemplary cell proliferative disorder include, but are not limited to, neoplasms, benign tumors, malignant tumors, pre-cancerous conditions, in situ tumors, encapsulated tumors, metastatic tumors, liquid tumors, solid tumors, immunological tumors, hematological tumors, cancers, carcinomas, leukemias, lymphomas, sarcomas, and rapidly dividing cells. The term “rapidly dividing cell” as used herein is defined as any cell that divides at a rate that exceeds or is greater than what is expected or observed among neighboring or juxtaposed cells within the same tissue. A cell proliferative disorder includes a precancer or a precancerous condition. A cell proliferative disorder includes cancer. A cell proliferative disorder includes a non-cancer condition or disorder. Preferably, the methods provided herein are used to treat or alleviate a symptom of cancer. The term “cancer” includes solid tumors, as well as, hematologic tumors, and/or malignancies. A “precancer cell” or “precancerous cell” is a cell manifesting a cell proliferative disorder that is a precancer or a precancerous condition. A “cancer cell” or “cancerous cell” is a cell manifesting a cell proliferative disorder that is a cancer. Any reproducible means of measurement may be used to identify cancer cells or precancerous cells. Cancer cells or precancerous cells can be identified by histological typing or grading of a tissue sample (e.g., a biopsy sample). Cancer cells or precancerous cells can be identified through the use of appropriate molecular markers.

As used herein, a “normal cell” is a cell that cannot be classified as part of a “cell proliferative disorder”, “cancer”, or “tumor”. A normal cell lacks unregulated or abnormal growth, or both, that can lead to the development of an unwanted condition or disease. Preferably, a normal cell possesses normally functioning cell cycle checkpoint control mechanisms.

Exemplary non-cancerous conditions or disorders include, but are not limited to, rheumatoid arthritis; inflammation; autoimmune disease; lymphoproliferative conditions; acromegaly; rheumatoid spondylitis; osteoarthritis; gout, other arthritic conditions; sepsis; septic shock; endotoxic shock; gram-negative sepsis; toxic shock syndrome; asthma; adult respiratory distress syndrome; chronic obstructive pulmonary disease; chronic pulmonary inflammation; inflammatory bowel disease; Crohn's disease; skin-related hyperproliferative disorders, psoriasis; eczema; atopic dermatitis; hyperpigmentation disorders, eye-related hyperproliferative disorders, age-related macular degeneration, ulcerative colitis; pancreatic fibrosis; hepatic fibrosis; acute and chronic renal disease; irritable bowel syndrome; pyresis; restenosis; cerebral malaria; stroke and ischemic injury; neural trauma; Alzheimer's disease; Huntington's disease; Parkinson's disease; acute and chronic pain; allergic rhinitis; allergic conjunctivitis; chronic heart failure; acute coronary syndrome; cachexia; malaria; leprosy; leishmaniasis; Lyme disease; Reiter's syndrome; acute synovitis; muscle degeneration, bursitis; tendonitis; tenosynovitis; herniated, ruptures, or prolapsed intervertebral disk syndrome; osteopetrosis; thrombosis; restenosis; silicosis; pulmonary sarcosis; bone resorption diseases, such as osteoporosis; graft-versus-host reaction; fibroadipose hyperplasia; spinocerebullar ataxia type 1; CLOVES syndrome; Harlequin ichthyosis; macrodactyly syndrome; Proteus syndrome (Wiedemann syndrome); LEOPARD syndrome; systemic sclerosis; Multiple Sclerosis; lupus; fibromyalgia; AIDS and other viral diseases such as Herpes Zoster, Herpes Simplex I or II, influenza virus and cytomegalovirus; diabetes mellitus; hemihyperplasia-multiple lipomatosis syndrome; megalencephaly; rare hypoglycemia, Klippel-Trenaunay syndrome; harmatoma; Cowden syndrome; or overgrowth-hyperglycemia.

Exemplary cancers include, but are not limited to, adrenocortical carcinoma, AIDS-related cancers, AIDS-related lymphoma, anal cancer, anorectal cancer, cancer of the anal canal, anal squamous cell carcinoma, angiosarcoma, appendix cancer, childhood cerebellar astrocytoma, childhood cerebral astrocytoma, basal cell carcinoma, skin cancer (non-melanoma), biliary cancer, extrahepatic bile duct cancer, intrahepatic bile duct cancer, bladder cancer, urinary bladder cancer, bone and joint cancer, osteosarcoma and malignant fibrous histiocytoma, brain cancer, brain tumor, brain stem glioma, cerebellar astrocytoma, cerebral astrocytoma/malignant glioma, ependymoma, medulloblastoma, supratentorial primitive neuroectodeimal tumors, visual pathway and hypothalamic glioma, breast cancer, bronchial adenomas/carcinoids, carcinoid tumor, gastrointestinal, nervous system cancer, nervous system lymphoma, central nervous system cancer, central nervous system lymphoma, cervical cancer, childhood cancers, chronic lymphocytic leukemia, chronic myelogenous leukemia, chronic myeloproliferative disorders, colon cancer, colorectal cancer, cutaneous T-cell lymphoma, lymphoid neoplasm, mycosis fungoides, Seziary Syndrome, endometrial cancer, esophageal cancer, extracranial germ cell tumor, extragonadal germ cell tumor, extrahepatic bile duct cancer, eye cancer, intraocular melanoma, retinoblastoma, gallbladder cancer, gastric (stomach) cancer, gastrointestinal carcinoid tumor, gastrointestinal stromal tumor (GIST), germ cell tumor, ovarian germ cell tumor, gestational trophoblastic tumor glioma, head and neck cancer, head and neck squamous cell carcinoma, hepatocellular (liver) cancer, Hodgkin lymphoma, hypopharyngeal cancer, intraocular melanoma, ocular cancer, islet cell tumors (endocrine pancreas), Kaposi Sarcoma, kidney cancer, renal cancer, kidney cancer, laryngeal cancer, acute lymphoblastic leukemia, T-cell lymphoblastic leukemia, acute myeloid leukemia, chronic lymphocytic leukemia, chronic myelogenous leukemia, hairy cell leukemia, lip and oral cavity cancer, liver cancer, lung cancer, non-small cell lung cancer, small cell lung cancer, lung squamous cell carcinoma, AIDS-related lymphoma, non-Hodgkin lymphoma, primary central nervous system lymphoma, B-cell lymphoma, primary effusion lymphoma, Waldenstram macroglobulinemia, medulloblastoma, melanoma, intraocular (eye) melanoma, merkel cell carcinoma, lewis cell carcinoma, mesothelioma malignant, mesothelioma, metastatic squamous neck cancer, mouth cancer, cancer of the tongue, multiple endocrine neoplasia syndrome, mycosis fungoides, myelodysplastic syndromes, myelodysplastic/myeloproliferative diseases, chronic myelogenous leukemia, acute myeloid leukemia, multiple myeloma, chronic myeloproliferative disorders, nasopharyngeal cancer, neuroblastoma, oral cancer, oral cavity cancer, oropharyngeal cancer, ovarian cancer, ovarian epithelial cancer, ovarian low malignant potential tumor, pancreatic cancer, islet cell pancreatic cancer, pancreatic endocrine tumor, paranasal sinus and nasal cavity cancer, parathyroid cancer, cholangiocarcinoma, penile cancer, pharyngeal cancer, pheochromocytoma, pineoblastoma and supratentorial primitive neuroectodermal tumors, pituitary tumor, pituitary adenoma, plasma cell neoplasm/multiple myeloma, pleuropulmonary blastoma, prostate cancer, rectal cancer, renal pelvis and ureter, transitional cell cancer, retinoblastoma, rhabdomyosarcoma, salivary gland cancer, Ewing family of sarcoma tumors, Kaposi Sarcoma, soft tissue sarcoma, uterine cancer, uterine sarcoma, skin cancer (non-melanoma), skin cancer (melanoma), merkel cell skin carcinoma, small intestine cancer, soft tissue sarcoma, squamous cell carcinoma, stomach (gastric) cancer, supratentorial primitive neuroectodermal tumors, testicular cancer, throat cancer, thymoma, thymoma and thymic carcinoma, thyroid cancer, transitional cell cancer of the renal pelvis and ureter and other urinary organs, gestational trophoblastic tumor, urethral cancer, endometrial uterine cancer, uterine sarcoma, uterine corpus cancer, vaginal cancer, vulvar cancer, and Wilm's Tumor.

In particular embodiments, the cancer is attacked by T cell mediated immunity. In specific embodiments, the cancer is lymphoma, melanoma, colon carcinoma, mammary carcinoma, lung carcinoma, fibrosarcoma, renal carcinoma, neuroblastoma, or ovarian carcinoma. For example, the cancer can be a lymphoma tumor, melanoma tumor, colon carcinoma tumor, mammary carcinoma tumor, lung carcinoma tumor, fibrosarcoma tumor, renal carcinoma tumor, neuroblastoma tumor, ovarian carcinoma tumor, or Lewis cell carcinoma (Lewis lung carcinoma).

A cancer that is to be treated can be staged according to the American Joint Committee on Cancer (AJCC) TNM classification system, where the tumor (T) has been assigned a stage of TX, T1, T1mic, T1a, T1b, T1c, T2, T3, T4, T4a, T4b, T4c, or T4d; and where the regional lymph nodes (N) have been assigned a stage of NX, N0, N1, N2, N2a, N2b, N3, N3a, N3b, or N3c; and where distant metastasis (M) can be assigned a stage of MX, M0, or M1. A cancer that is to be treated can be staged according to an American Joint Committee on Cancer (AJCC) classification as Stage I, Stage IIA, Stage IIB, Stage IIIA, Stage IIIB, Stage IIIC, or Stage IV. A cancer that is to be treated can be assigned a grade according to an AJCC classification as Grade GX (e.g., grade cannot be assessed), Grade 1, Grade 2, Grade 3 or Grade 4. A cancer that is to be treated can be staged according to an AJCC pathologic classification (pN) of pNX, pNO, PN0 (I−), PN0 (I+), PN0 (mol−), PN0 (mol+), PN1, PN1(mi), PN1a, PN1b, PN1c, pN2, pN2a, pN2b, pN3, pN3a, pN3b, or pN3c.

A cancer that is to be treated can be classified by microscopic appearance as well differentiated, moderately differentiated, poorly differentiated, or undifferentiated. A cancer that is to be treated can be classified by microscopic appearance with respect to mitosis count (e.g., amount of cell division) or nuclear pleiomorphism (e.g., change in cells). A cancer that is to be treated can be classified by microscopic appearance as being associated with areas of necrosis (e.g., areas of dying or degenerating cells). A cancer that is to be treated can be classified as having an abnormal karyotype, having an abnormal number of chromosomes, or having one or more chromosomes that are abnormal in appearance. A cancer that is to be treated can be classified as being aneuploid, triploid, tetraploid, or as having an altered ploidy. A cancer that is to be treated can be classified as having a chromosomal translocation, or a deletion or duplication of an entire chromosome, or a region of deletion, duplication or amplification of a portion of a chromosome.

As used herein, a “subject” or “subject in need thereof” is a subject having a cell proliferative disorder, or cancer or a subject having an increased risk of developing a cell proliferative disorder or cancer relative to the population at large. A subject in need thereof can have a precancerous condition. In specific embodiments, a subject in need thereof has cancer. A “subject” includes a mammal. The mammal can be e.g., any mammal, e.g., a human, primate, bird, mouse, rat, fowl, dog, cat, cow, horse, goat, rabbit, camel, sheep or a pig. In certain embodiments, the mammal is a human. In some embodiments, the human undergoing treatment can be a newborn, infant, toddler, preadolescent, adolescent, or adult.

In particular embodiments, the subject has normal or increased expression of a LAP-related molecule compared to the expression of the corresponding expression of the LAP-related molecule in a proper control, such as the population as a whole. In particular embodiments, normal or increased expression of a LAP-related molecule can be detected in the microenvironment of the tumor.

Reduction of LAP activity or reduction of the activity or expression of a LAP related molecule can skew CD4+ T cells toward a T-helper 1 (Th1) cellular response phenotype in the tumor microenvironment. For example, the Th1 phenotype can be increased by at least about 5%, 10%, 20%, 30%, 40%, 50%, 60%, 70%, 80%, 90%, 100%, 150%, 200% or greater increase in the anti-inflammatory cytokine level. Such increases can also include, for example, at least about a 3%-15%, 10%-25%, 20% to 35%, 30% to 45%, 40%-55%, 50%-65%, 60%-75%, 70%-85%, 80%-95%, 90%-105%, 100%-115%, 105%-120%, 115%-130%, 125%-150%, 140%-160%, 155%-500% or greater increase in the Th1 phenotype compared to the Th1 phenotype prior to administration of a LAP inhibitor. The Th1 cellular response can be measured by any means in the art including the Enzyme-linked immunosorbent spot (“ELISPOT”) assay for INFγ or intracellular cytokine staining for INFγ and TNFα. Accordingly, INFγ and TNFα can be increased following administration of a LAP-inhibitor. The expression of INFγ and TNFα can be increased by at least about 5%, 10%, 20%, 30%, 40%, 50%, 60%, 70%, 80%, 90%, 100%, 150%, 200% or greater when compared to expression prior to administration of the LAP inhibitor. In some embodiments, the Th1 cells are capable of secreting cytokines selected from the group of interferon gamma, interleukin 2, and TNF-beta. In some embodiments, the Th1 cells express markers that are selected from the group of CD4, CD94, CD119 (IFNγ R1), CD183 (CXCR3), CD186 (CXCR6), CD191 (CCR1), CD195 (CCR5), CD212 (IL-12R.beta.1&2), CD254 (RANKL), CD278 (ICOS), IL-18R, MRP1, NOTCH3, and TIM3.

There is mounting evidence that myeloid cells at the tumor microenvironment (TME), including M2 macrophages, N2 neutrophils and myeloid-derived suppressor cells (MDSC), play a critical role in immune suppression by directly causing functional exhaustion of the cytotoxic T cells or by indirectly increasing the suppressive power of T-regulatory cells (Tregs). This leads to a skewed immunostimulatory versus immunosuppressive balance in the TME. The immunostimulatory environment of the TME is largely shaped by the presence of cytotoxic T cells and NK cells, cytolytic and phagocytosis-inducing M1 macrophages, cytotoxic N1 neutrophils, humoral response inducing B cells, and antigen presenting immunogenic dendritic cells (DC). Immunostimulatory cytokines and chemokines such as interferon gamma (IFN-.gamma.), interleukin-12 (IL-12), tumor necrosis factor-alpha (TNF-.alpha.), etc. are key coordinators of the immunostimulatory activity. Important players that bias the immunosuppressive nature of the TME are anti-inflammatory Th2 cells, N2 neutrophils, M2 macrophages, Tregs, and tolerogenic DC. Immunosuppressive cytokines and chemokines such as transforming growth factor-beta (TGF-.beta.), interleukin-10 (IL-10), macrophage colony stimulating factor (M-CSF), interleukin-4 (IL-4), etc. are key coordinators of the immunosuppressive activity. In specific embodiments, the Th1 response in a tumor microoenvironment is increased by administration of a LAP inhibitor.

As described herein, the “tumor microenvironment” (TME) is the surrounding microenvironment that constantly interacts with tumor cells which is conducive to allow cross-talk between tumor cells and its environment. A tumor microenvironment plays a role in disrupting the cancer immunity cycle and plays a critical role in multiple aspects of cancer progression. For example, the TME can decrease drug penetration, confer proliferative and anti-apoptotic advantages to surviving cells, facilitate resistance without causing genetic mutations and epigenetic changes, and collectively modify disease modality and distort clinical indices. Without being limiting, the tumor microenvironment can include the cellular environment of the tumor, surrounding blood vessels, immune cells, fibroblasts, bone marrow derived inflammatory cells, lymphocytes, signaling molecules and the extracellular matrix. The tumor environment can include tumor cells or malignant cells that are aided and influenced by the tumor microenvironment to ensure growth and survival. The tumor microenvironment can also include tumor-infiltrating immune cells such as lymphoid and myeloid cells, which can stimulate or inhibit the antitumor immune response and stromal cells such as tumor-associated fibroblasts and endothelial cells that contribute to the tumor's structural integrity. Without being limiting, stromal cells can include cells that make up tumor-associated blood vessels, such as endothelial cells and pericytes, which are cells that contribute to structural integrity (fibroblasts), as well as tumor-associated macrophages (TAMs) and infiltrating immune cells including monocytes, neutrophils (PMN), dendritic cells (DCs), T and B cells, mast cells, and natural killer (NK) cells. The stromal cells make up the bulk of tumor cellularity while the dominating cell type in solid tumors is the macrophage.

Reduction of LAP activity or reduction of the activity or expression of a LAP related molecule can increase M1 macrophage polarization. For example, the M1 macrophage production can be increased by at least about 5%, 10%, 20%, 30%, 40%, 50%, 60%, 70%, 80%, 90%, 100%, 150%, 200% or greater when compared to the M1 macrophage level prior to administration of a LAP inhibitor.

Macrophages may be classified by subsets: classically (M1) or alternatively (M2) activated macrophages (see, e.g., Laskin, Chem Res Toxicol. 2009 Aug. 17; 22(8): 1376-1385, the contents of which are hereby incorporated by reference in their entireties). Without wishing to be bound by theory, M1 macrophages are activated by standard mechanisms, such as IFNγ, LPS, and TNFα, while M2 macrophages are activated by alternative mechanisms, such as IL-4, IL-13, IL-10, and TGFβ. M1 macrophages can display a cytotoxic, proinflammatory phenotype, while M2 macrophages, suppress some aspects of immune and inflammatory responses and participate in wound repair and angiogenesis. In some embodiments, administration of a LAP inhibitor can increase the level of M1 macrophages. In particular embodiments, administration of a LAP inhibitor can increase the level of M1 macrophages in the tumor microenvironment.

In certain embodiments, reduction of LAP activity or reduction of the activity or expression of a LAP related molecule can reduce pulmonary metastasis or micrometastases. For example, pulmonary metastasis or micrometastases can be reduced by about 5%, 10%, 20%, 30%, 40%, 50%, 60%, 70%, 80%, 90%, 100%, 150%, 200% or greater when compared to the pulmonary metastasis or micrometastases level prior to administration of a LAP inhibitor.

B. LAP Inhibitors

The methods and compositions disclosed herein encompass administration of an effective amount of a pharmaceutical composition that reduces LAP activity or reduces the activity or expression of a LAP related molecule. A composition or molecule that reduces LAP activity or reduces the activity or expression of a LAP related molecule could be any molecule that increases or decreases (i.e., modulates) LAP activity. As used herein, the term “specifically” means the ability of a molecule to reduce LAP activity or reduce the activity or expression of a LAP related molecule without impacting other related processes (i.e., canonical autophagy). A molecule that inhibits LAP activity preferentially, increases or decreases LAP activity, but might impact other phagocytosis-related pathways. Accordingly, a molecule that reduces LAP activity or reduces the activity or expression of a LAP related molecule could be any LAP-related nucleic acid, protein, or cytokine, such as Beclin1, VPS34, UVRAG, ATG5, ATG12, ATG16L, ATG7, ATG3, ATG4, LC3A, LC3B, GATE16, GABARAP, Rubicon, or NOX2.

For example, various embodiments of the present invention pertain to methods for modulating LAP activity which comprise administering to a cell an effective amount of an agent which reduces the biological activity of Rubicon and/or NOX2.

The LAP pathway relies on LAP-related molecules in order to recognize and clear dead cells. Accordingly, the expression or activity of any LAP-related molecule can be decreased in order to treat the cancer or cell proliferative disorder described herein. For example, the expression or activity of Beclin1, BPS34, UVRAG, ATG7, ATG3, ATG5, ATG12, ATG16L, ATG3, ATG4, LC3 family LC3A, LC3B, GATE16, GABARAP, Rubicon, NOX2, or any combination thereof can be reduced by any means in the art.

In some embodiments, expression or activity of the LAP-related molecule is reduced in any cell, such as a cancer and/or tumor cell, by using a small molecule inhibitor of Beclin1, BPS34, UVRAG, ATG7, ATG3, ATG5, ATG12, ATG16L, ATG3, ATG4, LC3 family LC3A, LC3B, GATE16, GABARAP, Rubicon, NOX2 or any combination thereof. As used herein, “small molecule inhibitors” include, but are not limited to, small peptides or peptide-like molecules, soluble peptides, and synthetic non-peptidyl organic or inorganic compounds. A small molecule inhibitor or antagonist can have a molecular weight of any of about 100 to about 20,000 daltons (Da), about 500 to about 15,000 Da, about 1000 to about 10,000 Da.

In some embodiments, expression or activity of the LAP-related molecule or LAP activity can be reduced by a LAP inhibitor acting by downregulating or reducing protein expression of Beclin1, BPS34, UVRAG, ATG7, ATG3, ATG5, ATG12, ATG16L, ATG3, ATG4, LC3 family LC3A, LC3B, GATE16, GABARAP, Rubicon, NOX2, or any combination thereof. Protein expression of a LAP-related molecule (i.e., LAP-related protein) can be reduced by any method known in the art for reducing protein expression. For example, protein expression can be reduced by using RNA interference such as siRNA or shRNA, by using antisense RNA, or by knocking out the gene encoding the LAP-related protein. In particular embodiments, protein expression or activity of a LAP-related protein can be reduced using an antisense nucleic acid, a ribozyme, a peptide, an antibody, an antagonist, an aptamer, or a peptidomimetic that reduces the expression or activity of a LAP-related protein respectively.

RNA interference (RNAi) is a phenomenon in which the introduction of double-stranded RNA (dsRNA) into a diverse range of organisms and cell types causes degradation of the complementary mRNA. In the cell, long dsRNAs are cleaved into short 21-25 nucleotide small interfering RNAs, or siRNAs, by a ribonuclease known as Dicer. The siRNAs subsequently assemble with protein components into an RNA-induced silencing complex (RISC), unwinding in the process. Activated RISC then binds to complementary transcript by base pairing interactions between the siRNA antisense strand and the mRNA. The bound mRNA is cleaved and sequence specific degradation of mRNA results in gene silencing. See, for example, U.S. Pat. No. 6,506,559.

The term “siRNA” as used herein refers to small interfering RNA, also known as short interfering RNA or silencing RNA. siRNAs can be, for example, 18 to 30, 20 to 25, 21 to 23 or 21 nucleotide-long double-stranded RNA molecules. A “shRNA” as used herein is a short hairpin RNA, which is a sequence of RNA that makes a (tight) hairpin turn that can also be used to silence gene expression via RNA interference. shRNA can by operably linked to the U6 promoter expression. The shRNA hairpin structure is cleaved by the cellular machinery into siRNA. shRNA disclosed herein can comprise a sequence complementary to at least 13 nucleotides, at least 14 nucleotides, at least 15 nucleotides, at least 16 nucleotides, at least 17 nucleotides, at least 18 nucleotides, at least 19 nucleotides, at least 20 nucleotides, at least 21 nucleotides, at least 22 nucleotides, or 23 nucleotides of the mRNA of a LAP-related protein.

Reduction (i.e., decreasing) of the expression of a LAP-related gene or protein can be achieved by any means known in the art. For example, gene expression can be decreased by a mutation. The mutation can be an insertion, a deletion, a substitution or a combination thereof, provided that the mutation leads to a decrease in the expression of the LAP-related protein. In specific embodiments recombinant DNA technology can be used to introduce a mutation into a specific site on the chromosome. Such a mutation may be an insertion, a deletion, a replacement of one nucleotide by another one or a combination thereof, as long as the mutated gene leads to a decrease in the expression of a LAP-related protein. Such a mutation can be made by deletion of a number of base pairs. In one embodiment, the deletion of one single base pair could render a gene encoding a LAP-related protein non-functional, thereby decreasing the expression of a LAP-related protein, since as a result of such a mutation, the other base pairs are no longer in the correct reading frame. In other embodiments, multiple base pairs are removed, such as about 2, 5, 10, 20, 30, 40, 50, 75, 100, 150, 200, 300, 400, 500, or more base pairs. In still other embodiments, the entire length of the gene encoding a LAP-related protein is deleted. Mutations introducing a stop-codon in the open reading frame, or mutations causing a frame-shift in the open reading frame could be used to reduce the expression of a gene encoding a LAP-related protein.

Other techniques for decreasing the expression of a gene encoding a LAP-related protein in order to reduce LAP activity and treat cancer as described herein are well-known in the art. For example, techniques may include modification of the gene by site-directed mutagenesis, restriction enzyme digestion followed by re-ligation, PCR-based mutagenesis techniques, allelic exchange, allelic replacement, RNA interference, or post-translational modification. Standard recombinant DNA techniques are all known in the art and described in Maniatis/Sambrook (Sambrook, J. et al. Molecular cloning: a laboratory manual. ISBN 0-87969-309-6). Site-directed mutations can be made by means of in vitro site directed mutagenesis using methods well known in the art.

Inhibitory molecules such as, inhibitory small molecules, nucleic acid molecules, such as siRNA or shRNA, ribozymes, peptides, antibodies, antagonist, aptamers, and peptidomimetics that reduces the expression or activity of a LAP-related protein can be introduced into primary eukaryotic cells using any method known in the art for introduction of molecules into eukaryotic cells. By “introducing” is intended presenting to the eukaryotic cell the expression cassette, mRNA, or polypeptide in such a manner that the sequence gains access to the interior of the primary eukaryotic cell. The methods provided herein do not depend on a particular method for introducing an expression cassette or sequence into a primary eukaryotic cell, only that the polynucleotide or polypeptide gains access to the interior of at least one primary eukaryotic cell. Methods for introducing sequences into eukaryotic cells are known in the art and include, but are not limited to, stable transformation methods, transient transformation methods, and virus-mediated methods.

The LAP inhibitor as described herein can be administered according to methods described herein in a variety of means known to the art. The LAP inhibitor can be used therapeutically either as exogenous materials or as endogenous materials. Exogenous agents are those produced or manufactured outside of the body and administered to the body. Endogenous agents are those produced or manufactured inside the body by some type of device (biologic or other) for delivery within or to other organs in the body. Administration can be parenteral, pulmonary, oral, topical, intradermal, intramuscular, intraperitoneal, intravenous, subcutaneous, intranasal, epidural, ophthalmic, buccal, or rectal administration.

Any LAP inhibitor as disclosed herein can be administered in a variety of methods well known in the arts. Administration can include, for example, methods involving oral ingestion, direct injection (e.g., systemic or stereotactic), implantation of cells engineered to secrete the factor of interest, drug-releasing biomaterials, polymer matrices, gels, permeable membranes, osmotic systems, multilayer coatings, microparticles, implantable matrix devices, mini-osmotic pumps, implantable pumps, injectable gels and hydrogels, liposomes, micelles (e.g., up to 30 μm), nanospheres (e.g., less than 1 μm), microspheres (e.g., 1-100 μm), reservoir devices, a combination of any of the above, or other suitable delivery vehicles to provide the desired release profile in varying proportions. Other methods of controlled-release delivery of agents or compositions will be known to the skilled artisan and are within the scope of the present disclosure.

Delivery systems may include, for example, an infusion pump which may be used to administer the LAP inhibitor in a manner similar to that used for delivering insulin or chemotherapy to specific organs or tumors. Typically, using such a system, LAP inhibitor can be administered in combination with a biodegradable, biocompatible polymeric implant that releases the agent over a controlled period of time at a selected site. Examples of polymeric materials include polyanhydrides, polyorthoesters, polyglycolic acid, polylactic acid, polyethylene vinyl acetate, and copolymers and combinations thereof. In addition, a controlled release system can be placed in proximity of a therapeutic target, thus requiring only a fraction of a systemic dosage.

LAP inhibitors can be encapsulated and administered in a variety of carrier delivery systems. Examples of carrier delivery systems include microspheres, hydrogels, polymeric implants, smart polymeric carriers, and liposomes (see generally, Uchegbu and Schatzlein, eds. (2006), Polymers in Drug Delivery, CRC, ISBN-10: 0849325331). Carrier-based systems for molecular or biomolecular agent delivery can: provide for intracellular delivery; tailor biomolecule/agent release rates; increase the proportion of biomolecule that reaches its site of action; improve the transport of the drug to its site of action; allow colocalized deposition with other agents or excipients; improve the stability of the agent in vivo; prolong the residence time of the agent at its site of action by reducing clearance; decrease the nonspecific delivery of the agent to non-target tissues; decrease irritation caused by the agent; decrease toxicity due to high initial doses of the agent; alter the immunogenicity of the agent; decrease dosage frequency, improve taste of the product; or improve shelf life of the product.

In specific embodiments, the expression or reduction of a LAP-related molecule, or LAP activity, is reduced by at least about 5%, 10%, 20%, 30%, 40%, 50%, 60%, 70%, 80%, 90%, 95%, 96%, 97%, 98%, 99%, 100%, 5-10%, 10-20%, 10-30%, 10-40%, 20-30%, 20-40%, 30-40%, 30-50%, 40-50%, 40-60%, 50-60%, 50-70%, 60-70%, 60-80%, 70-80%, 70-90%, 80-90%, 80-100%, 90-100%, or 95-100%. Gene or protein expression can be measured by any means known in the art.

The full-length amino acid sequence of murine Rubicon (GenBank accession number: AAH67390; gil45708948) has 941 amino acids, which is provided below, and is designated SEQ ID NO: 1.

SEQ ID NO: 1
MRPEGAGMDLGGGDGERLLEKSRREHWQLLGNLKTTVEGLVSANCPNVWS
KYGGLERLCRDMQNILYHGLIHDQVCCRQADYWQFVKDIRWLSPHSALHV
EKFISLHESD QSDTDSVSERAVAELWLQHSLQCHCLSAQLRPLLGDRQY
IRKFYTETAFLLSDAHVTAMLQCLEAVEQNNPRLLAQIDASMFARKQESP
LLVTKSQSLTALPGSTYTPPASYAQHSYFGSSSSLQSMPQSSHSSERRST
SFSLSGPSWQPQEDRECLSPAETQTTPAPLPSDSTLAQDSPLTAQEMSDS
TLTSPLEASWVSSQNDSPSDVSEGPEYLAIGNPAPHGRTASCESHSSNGE
SSSSHLFSSSSSQKLESAASSLGDQEEGRQSQAGSVLRRSSFSEGQTAPV
ASGTKKSHIRSHSDTNIASRGAAEGGQYLCSGEGMFRRPSEGQSLISYLS
EQDFGSCADLEKENAHFSISESLIAAIELMKCNMMSQCLEEEEVEEEDSD
REIQELKQKIRLRRQQIRTKNLLPAYRETENGSFRVTSSSSQFSSRDSTQ
LSESGSAEDADDLEIQDADIRRSAVSNGKSSFSQNLSHCFLHSTSAEAVA
MGLLKQFEGMQLPAASELEWLVPEHDAPQKLLPIPDSLPISPDDGQHADI
YKLRIRVRGNLEWAPPRPQIIFNVHPAPTRKIAVAKQNYRCAGCGIRTDP
DYIKRLRYCEYLGKYFCQCCHENAQMVVPSRILRKWDFSKYYVSNFSKDL
LLKIWNDPLFNVQDINSALYRKVKLLNQVRLLRVQLYHMKNMFKTCRLAK
ELLDSFDVVPGHLTEDLHLYSLSDLTATKKGELGPRLAELTRAGAAHVER
CMLCQAKGFICEFCQNEEDVIFPFELHKCRTCEECKACYHKTCFKSGRCP
RCERLQARRELLAKQSLESYLSDYEEEPTEALALEATVLETT

The full-length amino acid sequence of human Rubicon has 972 amino acids, which is provided below, and is designated SEQ ID NO: 2.

(SEQ ID NO: 2)
MRPEGAGMELGGGEERLPEESRREHWQLLGNLKTTVEGLVSTNSPNVWSK
YGGLERLCRDMQSILYHGLIRDQACRRQTDYWQFVKDIRWLSPHSALHVE
KFISVHENDQSSADGASERAVAELWLQHSLQYHCLSAQLRPLLGDRQYIR
KFYTDAAFLLSDAHVTAMLQCLEAVEQNNPRLLAQIDASMFARKHESPLL
VTKSQSLTALPSSTYTPPNSYAQHSYFGSFSSLHQSVPNNGSERRSTSFP
LSGPPRKPQESRGHVSPAEDQTIQAPPVSVSALARDSPLTPNEMSSSTLT
SPIEASWVSSQNDSPGDASEGPEYLAIGNLDPRGRTASCQSHSSNAESSS
SNLFSSSSSQKPDSAASSLGDQEGGGESQLSSVLRRSSFSEGQTLTVTSG
AKKSHIRSHSDTSIASRGAPESCNDKAKLRGPLPYSGQSSEVSTPSSLYM
EYEGGRYLCSGEGMFRRPSEGQSLISYLSEQDFGSCADLEKENAHFSISE
SLIAAIELMKCNMMSQCLEEEEVEEEDSDREIQELKQKIRLRRQQIRTKN
LLPMYQEAEHGSFRVTSSSSQFSSRDSAQLSDSGSADEVDEFEIQDADIR
RNTASSSKSFVSSQSFSHCFLHSTSAEAVAMGLLKQFEGMQLPAASELEW
LVPEHDAPQKLLPIPDSLPISPDDGQHADIYKLRIRVRGNLEWAPPRPQI
IFNVHPAPTRKIAVAKQNYRCAGCGIRTDPDYIKRLRYCEYLGKYFCQCC
HENAQMAIPSRVLRKWDFSKYYVSNFSKDLLIKIWNDPLFNVQDINSALY
RKVKLLNQVRLLRVQLCHMKNMFKTCRLAKELLDSFDTVPGHLTEDLHLY
SLNDLTATRKGELGPRLAELTRAGATHVERCMLCQAKGFICEFCQNEDDI
IFPFELHKCRTCEECKACYHKACFKSGSCPRCERLQARREALARQSLESY
LSDYEEEPAEALALEAAVLEAT

Rubicon protein is predicted to comprise a conserved RUN domain, near the N-terminus, a cysteine-rich domain at the C-terminus, and a coiled-coil domain (CCD) or motif in the central region. The predicted CCD of murine Rubicon has a sequence of amino acid sequences 488 to 508 of SEQ ID NO: 1. The predicted CCD of human Rubicon has a sequence of amino acid sequences 518 to 538 of SEQ ID NO: 2. One of ordinary skill in the art would understand how to generate a Rubicon polypeptide in view of the disclosure of SEQ ID NO: 1 and SEQ ID NO: 2 using any of a number of experimental methods well-known to those of skill in the art. In one embodiment, a Rubicon polypeptide having biological activity of a native Rubicon protein, the biological activity of a native Rubicon protein is as described in the examples, including, but not limited to, promoting the association of the active class III PI3K complex with the LAPosome and the production of PI(3)P (i.e., Rubicon activity) and stabilization of the active NOX2 complex to promote optimal ROS production.

The NADPH Oxidase (nicotinamide adenine dinucleotide phosphate-oxidase, Nox) family of enzymes emerged during the evolutionary transition from unicellular to multicellular organisms and catalyze the reduction of oxygen to superoxide. Nox2 is a member of the Nox family and is known by a variety of aliases, including CYBB (Cytochrome b-245, beta polypeptide (chronic granulomatous disease)). Aliases of Nox2 include: CYBB, AMCBX2; CGD; GP91-1; GP91-PHOX; GP91PHOX; and p91-PHOX. An exemplary amino acid sequence of Nox2 is provided in GenBank Accession No. NM_000397.3 (SEQ ID NO: 3) and GenBank Accession No. NP_031833.3 (SEQ ID NO: 4, murine Nox2). Nox2 is also referred to as the phagocytic “respiratory burst oxidase” for its role in the innate immune response, specifically in phagocyte killing of ingested microbes. In specific embodiments, the LAP inhibitor reduces Nox2 activity. For example, the LAP inhibitor can by apocynin, a Nox2 inhibitor. In particular embodiments the LAP inhibitor is o-methoxycatechol, an apocynin, a diapocynin, 4-(2-aminoethyl)-benzenesulfonyl fluoride (AEBSF), 4-hydroxy-3′-methoxy-acetophenon, N-Vanillylnonanamide, staurosporine or a combination thereof.

In some embodiments, the LAP inhibitor is an ROS scavenger. For example, in specific embodiments the LAP inhibitor is DPI. In some embodiments, the LAP inhibitor is a ROS scavenger, such as diphenyleneiodonium (DPI), apocynin, a procyanidin, Schisandrin B, or annexin peptide Ac2-26.

SEQ ID NO: 3
MGNWAVNEGLSIFVILVWLGLNVFLFVWYYRVYDIPPKFFYTRKLLGSAL
ALARAPAACLNFNCMLILLPVCRNLLSFLRGSSACCSTRVRRQLDRNLTF
HKMVAWMIALHSAIHTIAHLFNVEWCVNARVNNSDPYSVALSELGDRQNE
SYLNFARKRIKNPEGGLYLAVTLLAGITGVVITLCLILIITSSTKTIRRS
YFEVFWYTHHLFVIFFIGLAIHGAERIVRGQTAESLAVHNITVCEQKISE
WGKIKECPIPQFAGNPPMTWKWIVGPMFLYLCERLVRFWRSQQKVVITKV
VTHPFKTIELQMKKKGFKMEVGQYIFVKCPKVSKLEWHPFTLTSAPEEDF
FSIHIRIVGDWTEGLFNACGCDKQEFQDAWKLPKIAVDGPFGTASEDVFS
YEVVMLVGAGIGVTPFASILKSVWYKYCNNATNLKLKKIYFYWLCRDTHA
FEWFADLLQLLESQMQERNNAGFLSYNIYLTGWDESQANHFAVHHDEEKD
VITGLKQKTLYGRPNWDNEFKTIASQHPNTRIGVFLCGPEALAETLSKQS
ISNSESGPRGVHFIFNKENF
SEQ ID NO: 4
MGNWAVNEGLSIFVILVWLGLNVFLFINYYKVYDDGPKYNYTRKLLGSAL
ALARAPAACLNFNCMLILLPVCRNLLSFLRGSSACCSTRIRRQLDRNLTF
HKMVAWMIALHTAIHTIAHLFNVEWCVNARVGISDRYSIALSDIGDNENE
EYLNFAREKIKNPEGGLYVAVTRLAGITGIVITLCLILIITSSTKTIRRS
YFEVFWYTHHLFVIFFIGLAIHGAERIVRGQTAESLEEHNLDICADKIEE
WGKIKECPVPKFAGNPPMTWKWIVGPMFLYLCERLVRFWRSQQKVVITKV
VTHPFKTIELQMKKKGFKMEVGQYIFVKCPKVSKLEWHPFTLTSAPEEDF
FSIHIRIVGDWTEGLFNACGCDKQEFQDAWKLPKIAVDGPFGTASEDVFS
YEVVMLVGAGIGVTPFASILKSVWYKYCDNATSLKLKKIYFYWLCRDTHA
FEWFADLLQLLETQMQERNNANFLSYNIYLTGWDESQANHFAVHHDEEKD
VITGLKQKTLYGRPNWDNEFKTIASEHPNTTIGVFLCGPEALAETLSKQS
ISNSESGPRGVHFIFNKENF

In some embodiments, the LAP inhibitor comprises an inhibitor of the scavenging of reactive oxygen species (ROS). Thus, a LAP inhibitor can inhibit ROS scavenging by at least about 5%, 10%, 20%, 30%, 40%, 50%, 60%, 70%, 80%, 90%, 95%, 96%, 97%, 98%, 99%, 100%, 5-10%, 10-20%, 10-30%, 10-40%, 20-30%, 20-40%, 30-40%, 30-50%, 40-50%, 40-60%, 50-60%, 50-70%, 60-70%, 60-80%, 70-80%, 70-90%, 80-90%, 80-100%, 90-100%, or 95-100%. ROS scavenging can be measured by flow cytometry using the ROS probe H2DCFDA.

The pharmaceutical composition may be a liquid formulation or a solid formulation. When the pharmaceutical composition is a solid formulation it may be formulated as a tablet, a sucking tablet, a chewing tablet, a chewing gum, a capsule, a sachet, a powder, a granule, a coated particle, a coated tablet, an enterocoated tablet, an enterocoated capsule, a melting strip or a film. When the pharmaceutical composition is a liquid formulation it may be formulated as an oral solution, a suspension, an emulsion or syrup. Said composition may further comprise a carrier material independently selected from, but not limited to, the group consisting of lactic acid fermented foods, fermented dairy products, resistant starch, dietary fibers, carbohydrates, proteins, and glycosylated proteins. As used herein, the pharmaceutical composition could be formulated as a food composition, a dietary supplement, a functional food, a medical food, or a nutritional product as long as the required effect is achieved.

The pharmaceutical composition according to the invention, used according to the invention or produced according to the invention may also comprise other substances, such as an inert vehicle, or pharmaceutical acceptable adjuvants, carriers, preservatives etc., which are well known.

Generally, the dosage of the LAP inhibitor will vary depending upon such factors as the patient's age, weight, height, sex, general medical condition and previous medical history. In specific embodiments, it may be desirable to administer the LAP inhibitor in the range of from about 1 to 100 mg/kg, 20 to 30 mg/kg, 30 to 40 mg/kg, 40 to 50 mg/kg, 50 to 60 mg/kg, 60 to 70 mg/kg, 70 to 80 mg/kg, 80 to 100 mg/kg, 5 to 10 mg/kg, 2 to 10 mg/kg, 10 to 20 mg/kg, 5 to 15 mg/kg, 1 to 10 mg/kg, 1 to 5 mg/kg, 2 to 5 mg/kg or any range in between 1 and 100 mg/kg.

In some embodiments of the invention, the method comprises administration of multiple doses of the LAP inhibitor. The method may comprise administration of 1, 2, 3, 4, 5, 6, 7, 8, 9, 10, 15, 20, 25, 30, 35, 40, or more therapeutically effective doses of a LAP inhibitor. In some embodiments, doses are administered over the course of 1 day, 2 days, 3 days, 4 days, 5 days, 6 days, 7 days, 10 days, 14 days, 21 days, 30 days, or more than 30 days. The frequency and duration of administration of multiple doses of the compositions is such as to reduce the tumor size, tumor number, disease severity, or progression, and/or reduce LAP activity. Changes in dosage may result and become apparent from the results of diagnostic assays for detecting tumor size, tumor number, disease severity, or progression, and/or reduce LAP activity known in the art and described herein.

A “pharmaceutical composition” is a formulation containing the LAP inhibitor in a form suitable for administration to a subject. In one embodiment, the pharmaceutical composition is in bulk or in unit dosage form. The unit dosage form is any of a variety of forms, including, for example, a capsule, an IV bag, a tablet, a single pump on an aerosol inhaler or a vial. The quantity of active ingredient (e.g., a formulation of the disclosed compound or salt, hydrate, solvate or isomer thereof) in a unit dose of composition is an effective amount and is varied according to the particular treatment involved. One skilled in the art will appreciate that it is sometimes necessary to make routine variations to the dosage depending on the age and condition of the patient. The dosage will also depend on the route of administration. A variety of routes are contemplated, including oral, pulmonary, rectal, parenteral, transdermal, subcutaneous, intravenous, intramuscular, intraperitoneal, inhalational, buccal, sublingual, intrapleural, intrathecal, intranasal, and the like. Dosage forms for the topical or transdermal administration of a compound of this invention include powders, sprays, ointments, pastes, creams, lotions, gels, solutions, patches and inhalants. In one embodiment, the active compound is mixed under sterile conditions with a pharmaceutically acceptable carrier, and with any preservatives, buffers or propellants that are required. In specific embodiments the LAP inhibitor is administered as a solution, dispersion, suspension, powder, capsule, tablet, pill, time release capsule, time release tablet, and/or time release pill.

Moreover, the administration may be by continuous infusion or by single or multiple boluses. In specific embodiments, a LAP inhibitor can be infused over a period of less than about 4 hours, 3 hours, 2 hours or 1 hour. In still other embodiments, the infusion occurs slowly at first and then is increased over time.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required particle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol, sorbitol, sodium chloride in the composition. Prolonged absorption of the injectable compositions can be brought about by including in the composition an agent which delays absorption, for example, aluminum monostearate and gelatin.

Sterile injectable solutions can be prepared by incorporating the active compound in the required amount in an appropriate solvent with one or a combination of ingredients enumerated above, as required, followed by filtered sterilization. Generally, dispersions are prepared by incorporating the active compound into a sterile vehicle that contains a basic dispersion medium and the required other ingredients from those enumerated above. In the case of sterile powders for the preparation of sterile injectable solutions, methods of preparation are vacuum drying and freeze-drying that yields a powder of the active ingredient plus any additional desired ingredient from a previously sterile-filtered solution thereof.

Oral compositions generally include an inert diluent or an edible pharmaceutically acceptable carrier. They can be enclosed in gelatin capsules or compressed into tablets. For the purpose of oral therapeutic administration, the active compound can be incorporated with excipients and used in the form of tablets, troches, or capsules. Oral compositions can also be prepared using a fluid carrier for use as a mouthwash, wherein the compound in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or compounds of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the compounds are delivered in the form of an aerosol spray from pressured container or dispenser, which contains a suitable propellant, e.g., a gas such as carbon dioxide, or a nebulizer. Systemic administration can also be by transmucosal or transdermal means. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories. For transdermal administration, the active compounds are formulated into ointments, salves, gels, or creams as generally known in the art.

As used herein, “combination therapy” or “co-therapy” includes the administration of a LAP inhibitor, as part of a specific treatment regimen intended to provide the beneficial effect from the co-action of LAP inhibitor with an additional composition. The beneficial effect of the combination includes, but is not limited to, pharmacokinetic or pharmacodynamic co-action resulting from the combination of these at least two compounds of the present invention. Administration of these at least two compounds of the present invention in combination typically is carried out over a defined time period (usually minutes, hours, days or weeks depending upon the combination selected).

“Combination therapy” also embraces the administration of the LAP inhibitor as described herein in further combination with other biologically active ingredients and non-drug therapies (e.g., surgery or radiation treatment). Where the combination therapy further comprises a non-drug treatment, the non-drug treatment may be conducted at any suitable time so long as a beneficial effect from the co-action of the combination of the LAP inhibitor and non-drug treatment is achieved. For example, in appropriate cases, the beneficial effect is still achieved when the non-drug treatment is temporally removed from the administration of the LAP inhibitor, perhaps by days or even weeks.

In specific embodiments a LAP inhibitor can be administered in combination with a chemotherapeutic agent. The chemotherapeutic agent (also referred to as an anti-neoplastic agent or anti-proliferative agent) can be an alkylating agent; an antibiotic; an anti-metabolite; a detoxifying agent; an interferon; a polyclonal or monoclonal antibody; an EGFR inhibitor; an FGFR inhibitor, a HER2 inhibitor; a histone deacetylase inhibitor; a hormone; a mitotic inhibitor; an MTOR inhibitor; a multi-kinase inhibitor; a serine/threonine kinase inhibitor; a tyrosine kinase inhibitors; a VEGF/VEGFR inhibitor; a taxane or taxane derivative, an aromatase inhibitor, an anthracycline, a microtubule targeting drug, a topoisomerase poison drug, an inhibitor of a molecular target or enzyme (e.g., a kinase inhibitor), a cytidine analogue drug or any chemotherapeutic, anti-neoplastic or anti-proliferative agent.

Exemplary alkylating agents include, but are not limited to, cyclophosphamide (Cytoxan; Neosar); chlorambucil (Leukeran); melphalan (Alkeran); carmustine (BiCNU); busulfan (Busulfex); lomustine (CeeNU); dacarbazine (DTIC-Dome); oxaliplatin (Eloxatin); carmustine (Gliadel); ifosfamide (Ifex); mechlorethamine (Mustargen); busulfan (Myleran); carboplatin (Paraplatin); cisplatin (CDDP; Platinol); temozolomide (Temodar); thiotepa (Thioplex); bendamustine (Treanda); or streptozocin (Zanosar).In some embodiments, the additional chemotherapeutic agent can be a cytokine such as G-CSF (granulocyte colony stimulating factor).

In particular embodiments, a LAP inhibitor as disclosed herein can be administered in combination with radiation therapy. Radiation therapy can also be administered in combination with a LAP inhibitor and another chemotherapeutic agent described herein as part of a multiple agent therapy. In yet another aspect, a LAP inhibitor, may be administered in combination with standard chemotherapy combinations such as, but not restricted to, CMF (cyclophosphamide, methotrexate and 5-fluorouracil), CAF (cyclophosphamide, adriamycin and 5-fluorouracil), AC (adriamycin and cyclophosphamide), FEC (5-fluorouracil, epirubicin, and cyclophosphamide), ACT or ATC (adriamycin, cyclophosphamide, and paclitaxel), rituximab, Xeloda (capecitabine), Cisplatin (CDDP), Carboplatin, TS-1 (tegafur, gimestat and otastat potassium at a molar ratio of 1:0.4:1), Camptothecin-11 (CPT-11, Irinotecan or Camptosar) or CMFP (cyclophosphamide, methotrexate, 5-fluorouracil and prednisone).

### EMBODIMENTS

1. A method for treating cancer in a subject, said method comprising administering an effective amount of a LC3-associated phagocytosis (LAP) inhibitor to the subject.

2. The method of embodiment 1, wherein the LAP inhibitor reduces the expression or activity of at least one gene selected from the group consisting of: Beclin1, VPS34, UVRAG, ATG5, ATG12, ATG16L, ATG7, ATG3, ATG4, LC3A, LC3B, GATE16, GABARAP, Rubicon, and NOX2 is reduced.

3. The method of embodiment 1 or 2, wherein the LAP inhibitor reduces the expression or activity of ATG5.

4. The method of any one of embodiments 1-3, wherein said cancer is a cancer attacked by T-cell mediated immunity.

5. The method of any one of embodiments 1-4, wherein said cancer is acute myeloid gliomas, histiocytic lymphoma, non-Hodgkin's lymphoma, thyroid cancer, papillary thyroid carcinoma, head and neck cancer, liver cancer, colorectal cancer, pancreatic cancer, breast cancer, ovarian cancer, lung cancer, carcinoma, lymphoma, blastoma, medulloblastoma, retinoblastoma, sarcoma, liposarcoma, synovial cell sarcoma, neuroendocrine tumors, carcinoid tumors, gastrinoma, islet cell cancer, mesothelioma, schwannoma, acoustic neuroma, meningioma, adenocarcinoma, leukemia or lymphoid malignancies, squamous cell cancer, epithelial squamous cell cancer, small-cell lung cancer (SCLC), non-small cell lung cancer (NSCLC), adenocarcinoma of the lung, squamous carcinoma of the lung, cancer of the peritoneum, hepatocellular cancer, gastric or stomach cancer, gastrointestinal cancer, glioblastoma, cervical cancer, bladder cancer, hepatoma, metastatic breast cancer, colon cancer, rectal cancer, endometrial or uterine carcinoma, salivary gland carcinoma, kidney or renal cancer, prostate cancer, vulval cancer, hepatic carcinoma, anal carcinoma, penile carcinoma, Merkel cell cancer, mycoses fungoids, testicular cancer, esophageal cancer, tumors of the biliary tract, or hematological malignancies.

6. The method of any one of embodiments 1-5, wherein said effective amount of a LAP inhibitor is administered by inhalation, intranasally, orally, intravenously, subcutaneously, or intramuscularly.

7. The method of any one of embodiments 1-6, wherein the effective amount of a LAP inhibitor is administered to the subject in need thereof, in the form of a solution, dispersion, suspension, powder, capsule, tablet, pill, time release capsule, time release tablet, and time release pill.

8. The method of any one of embodiments 1-7, further comprising treating the subject with radiation before, during, or after the administration of the LAP inhibitor

9. The method of any one of embodiments 1-8, further comprising administering a chemotherapeutic agent to said subject.

10. The method of any one of embodiments 1-9, wherein said cancer comprises at least one tumor, and wherein the number of said tumors is reduced following administration of the LAP inhibitor compared to the number of tumors prior to administration of the LAP inhibitor.

11. The method of any one of embodiments 1-10, wherein said cancer comprises a tumor, wherein the size of said tumor is reduced following administration of the LAP inhibitor compared to the size of the tumor prior to administration of the LAP inhibitor.

12. The method of embodiment 10 or 11, wherein said tumor is a melanoma tumor or a Lewis cell carcinoma.

13. The method of any one of embodiments 1-12, wherein administration of the LAP inhibitor reduces the spread or delays the progression of said cancer.

14. The method of any one of embodiments 1-13, wherein administration of the LAP inhibitor increases the Th1 response in the subject.

15. The method of embodiment 14, wherein said subject has a tumor and wherein the Th1 response is increased in the tumor microenvironment.

16. The method of any one of embodiments 1-15, wherein expression of IFNγ and/or TNFα is increased following administration of the LAP inhibitor.

17. The method of embodiment 14, wherein said subject has a tumor and wherein the IFNγ and/or TNFα expression is increased in the tumor microenvironment following administration of the LAP inhibitor.

18. The method of any one of embodiments 1-17, wherein the M1 macrophage level is increased following administration of the LAP inhibitor.

19. The method of embodiment 18, wherein said subject has a tumor and wherein the M1 macrophage level is increased in the tumor microenvironment following administration of the LAP inhibitor.

20. The method of any one of embodiments 1-19 wherein tumor metastasis is reduced following administration of the LAP inhibitor.

21. The method of embodiment 20, wherein said tumor metastasis is pulmonary metastasis.

22. The method of any one of embodiments 1-21, wherein said LAP inhibitor reduces scavenging of reactive oxygen species.

23. The method of embodiment 22, wherein said LAP inhibitor is DPI.

24. The method of any one of embodiments 1-22, wherein said LAP inhibitor is apocynin.

25. Use of a LAP inhibitor for treating cancer in a subject, said use comprising administering an effective amount of a LC3-associated phagocytosis (LAP) inhibitor to the subject.

26. Use of a LAP inhibitor according to the method of any one of embodiments 1-24.

27. A LAP inhibitor for use in treating cancer in a subject, said use comprising administering an effective amount of a LC3-associated phagocytosis (LAP) inhibitor to the subject.

### EXPERIMENTAL

### Example 1. LAP-Deficiency in Myeloid Cells is Protective in a Graft Tumor Model

Mice: Atg7^{floxflox} mice were provided by M. Komatsu and bred to LysM-Cre⁺ mice (provided by P. Murray). LysM-Cre⁺ Atg5^{floxflox} were provided by T. A. Ferguson and LysM-Cre⁺ Fip200^{floxflox} were provided by J.-L. Guan. The St. Jude. Institutional Animal Care and Use Committee approved all procedures in accordance with the Guide for the Care and Use of Animals.

Media and reagents: Hanks solution, phosphate buffer and DMEM basal media were purchased from Gibco. DNase I was from Worthington, Liberase™ was from Roche and β-mercaptoethanol, ionomycin and PMA from Sigma. Fc block was from BD Biosciences. BV711 NK1.1, BV570 CD11b, PE-Cy7 CD206, APC-Fire 750 F4/80, PE-Cy7 Ly-6G anti-mouse antibodies and Zombie Violet fixable dead stain reagent were purchased from Biolegend. V500 CD45, PerCP Cy-5.5 Ly-6G, V450 CD11c, PerCP B220 anti-mouse antibodies were purchased from BD Biosciences. APC F4/80, APC-eFluor780 CD4, eFluor450 CD8, PerCP Cy-5.5 IFN-γ anti-mouse antibodies were purchased from eBiosciences. Anti-mouse CD4 (clone GK1.5), anti-mouse CD8α (clone 2.43) and rat IgG 2b isotype control (anti-KLH clone LTF-20) were purchased from BioCell.

Tumor models: 2.5×10⁵ B16F10 cells and 1.0×10⁵ LLC/2 cells (ATCC) resuspended in sterile phosphate buffer (PBS) were injected subcutaneously in the rear flank of 6-8 weeks old C57BL/6 Fip200^{flox/flox} LysM-Cre^{+/-}, Atg5^{flox/flox} LysM-Cre^{+/-} and Atg7^{flox/flox} LysM-Cre^{+/-} and LysM-Cre^{-/-} littermate controls. Tumor greatest longitudinal and transversal diameters were estimated with a digital caliper and tumor volume were calculated according to the modified ellipsoidal formula. For lung metastasis studies, 1.0×10⁵ B16F10 cells were resuspended in PBS, passed through a 70 μM strainer and injected in the tail vein of 10-12 weeks old mice. Mice were euthanized 21 days after the injection, the lungs were excised, washed in phosphate buffer and the number of metastatic spots in all the lobes were counted.

Isolation of tumor-infiltrating cells: Subcutaneous mouse tumors were removed 14 days after injection, minced and incubated in dissociation media (Hanks buffer supplemented with 1 mg/ml DNase I, and 100 μg/ml Liberase™) for 30 min at 37° C. incubator for digestion. Tissues were manually dissociated by resuspension in a DNase solution (DMEM containing 10% fetal bovine serum, β-mercaptoethanol and 1 mg/ml DNAse I). Cells were washed, and collected at the 40%-80% fraction of a Percoll gradient.

Flow cytometry: Single cell suspensions were blocked with Fc block (1:200) for 5 min at room temperature. Staining was carried out in PBS 1% bovine serum albumin 1 □M EDTA for 10 min on ice. For IFN-γ staining, single cell suspensions were incubated for 4 h at 37° C. 5 CO₂ with PMA (10 ng/ml) and ionomycin (1 μg/ml). Cells were fixed and stained with fixation/permeabilization kit (eBioscience) following manufacture's protocol. Flow analysis was performed on a Sony SP6800 spectra analyzer.

Cell Sorting: CD11b⁺ tumor-infiltrating cells were isolated by Percoll gradient, as mentioned above, followed by magnetic separation kit (MACS), following manufacturer's instructions. Purified cells were stained with antibodies against Ly-6G and F4/80 and tumor-associated macrophages sorting was carried out on Ly-6G⁻/F4/80⁺ population. Sorting was performed using a iCite Reflection system.

Quantitative PCR (qRT-PCR): RNA from tumor-associated macrophages were collected and processed using RNeasy (Qiagen) following manufacturer's instructions. cDNA was synthesized using SuperScript II (Invitrogen) and analyzed by qRT-PCR using Syber Green (Invitrogen) and the following primer pairs:

| | | |
|---|---|---|
| GAPDH | AGGTCGGTGTGAACGGATTTG | TGTAGACCATGTAGTTGAGGT |
| | | CA |
| IL-1β | GCAACTGTTCCTGAACTCAAC | ATCTTTTGGGGTCCGTCAACT |
| | T | |
| TNF-α | AGGAGGAGTCTGCGAAGAAGA | GGCAGTGGACCATCTAACTCG |
| TGF-β | CTCCCGTGGCTTCTAGTGC | GCCTTAGTTTGGACAGGATCT |
| | | G |
| IL-10 | GCTCTTACTGACTGGCATGAG | CGCAGCTCTAGGAGCATGTG |

T cell depletion: Mice were intraperitoneally injected with 150 μg of antibody (diluted in 1 mg/ml in sterile phosphate buffer), the day before and at days 4 and 9 after subcutaneous injection of 10⁵ LLC cells. Cell depletion was confirmed in blood-purified cells by flow cytometry at days 7 and 14 after tumor cells implantation.

### Example 2. Sample Methods for Measuring LAP Activity

As described in Martinez, J. et al. (Molecular characterization of LC3-associated phagocytosis (LAP) reveals distinct roles for Rubicon, NOX2, and autophagy proteins. Nature cell biology, 17, 893-906.), multiple methods can be used to measure LAP activity.

Cell lysis and immunoblotting. Cells can be lysed in RIPA buffer for 30 min on ice (50 mM Tris, pH 7.5, 150 mM NaCl, 1% Triton X-100, 0.5% DOC, 0.1% SDS, protease inhibitor tablet (Roche), 1 mM NaF, 1 mM Na3VO4 and 1 mM phenylmethylsulphonyl fluoride). After centrifugation (16.1 k rcf, 15 min, 4° C.), supernatants can be analysed by SDS-PAGE. Anti-LC3B (catalogue no. ab48394) and anti-UNC93B (catalogue no. ab69497) antibodies can be from abCam. Anti-GATE16 (clone EP4808, catalogue no. TA310512) antibody can be from Origene. Anti-Actin antibody (clone C4, catalogue no. 08691001) can be from MP Biomedicals. Anti-ATG7 (clone D12B11, catalogue no. 8558), anti-Beclin1 (clone D40C5, catalogue no. 3495), anti-UVRAG (clone D2Q1Z, catalogue no. 13115), anti-VPS34 (clone D9A5, catalogue no. 4263), anti-Rubicon (clone D9F7, catalogue no. 8465), anti-p-p40PHOX (catalogue no. 4311), anti-ATG14 (catalogue no. 5504), anti-LC3A (clone D50G8, catalogue no. 4599) and anti-GABARAP (clone E1J4E, catalogue no. 13733) antibodies can be from Cell Signaling. p22PHOX (clone C17, catalogue no. 11712) antibody can be from Santa Cruz Biotechnology. Anti-RAB5 (catalogue no. R4654) and anti-RAB7 (catalogue no. R4779) antibodies can be from Sigma-Aldrich. All primary antibodies, except anti-Actin, were used at a 1:1,000 dilution. Anti-actin antibody was used at 1:10,000. All HRP-conjugated secondary antibodies can be used at a 1:2,000 dilution.

Phagosomes from BMDMs and RAW cells can be obtained as previously described. Briefly, after culture of cells with Pam3csk4-coupled beads, the cells can be washed in cold PBS, pelleted, resuspended in 1 ml of homogenization buffer (250 mM sucrose, 3 mM imidazole, pH 7.4), and homogenized on ice in a Dounce homogenizer. Phagosomes can be isolated by flotation on a sucrose step gradient during centrifugation for 1 h at 100,000 g at 4 C. The latex-bead phagosomal fraction was then collected from the interface of the 10% and 25% sucrose solutions and resuspended in RIPA buffer for protein immunoblot analysis. The entire phagosome purification can be run on 1-2 SDS-PAGE gels owing to the relatively lower protein yield compared with whole-cell lysate samples. Membranes can be sectioned according to the molecular weight marker, and proteins residing within that range of molecular weights were probed with the antibodies listed above. When necessary, membranes can be stripped with Restore PLUS Western Blot Stripping Buffer (Life Technologies), re-blocked in 1× TBST with 5% w/v non-fat dry milk, and probed with fresh antibodies. Images can be captured with an Amersham Imager 600.

Time-lapse imaging and microscopy. Cells can be plated on fibronectin-coated glass-bottom chamber slides (MatTek). Confocal microscopy can be performed using the following systems. Spinning-disc confocal microscopy (SDC) on live cells can be performed with a Marianas SDC imaging system (Intelligent Imaging Innovations/3i) consisting of a CSU22 confocal head (Yokogawa Electric Corporation), DPSS lasers (CrystaLaser) with wavelengths of 445 nm, 473 nm, 523 nm, 561 nm and 658 nm, and a Carl Zeiss 200M motorized inverted microscope (Carl Zeiss MicroImaging), equipped with spherical aberration correction optics (3i). Temperature can be maintained at ˜37 C and 5% CO2 using an environmental control chamber (Solent Scientific). Images can be acquired with a Zeiss Plan-Neofluar 40×1.3 NA DIC objective on a Cascadell 512 EMCCD (Photometrics), using SlideBook 6 software (3i).

Laser scanning confocal microscopy (LSCM) on live cells can be performed with a Nikon TE2000-E inverted microscope equipped with a ClSi confocal system, (Nikon), an argon ion laser at 488 nm and DPSS lasers at 404 nm and 561 nm (Melles Griot). Temperature can be maintained at 37 C and 5% CO2 using an environmental control chamber (InVivo Scientific). Images can be taken at the intervals indicated in the figure legends using an oil-immersion Nikon Plan Fluor 40×1.3 NA objective with phase contrast optics.

Flow cytometry analysis. At the indicated time points, GFP-LC3+ cells can be collected, washed once with FACS buffer, and permeabilized with digitonin (Sigma, 200 g ml-1) for 15 min on ice. Cells can be washed 3 times with FACS buffer and analysed by flow cytometry for membrane-bound GFP-LC3-II. Likewise, PX-mCherry+ cells can be collected, washed once with FACS buffer, and treated with digitonin (200 g ml-1) for 15 min on ice. Cells can then be washed 3 times with FACS buffer and analysed by flow cytometry for membrane-bound PtdIns(3)P.

Quantification of phagocytosis. Phagocytosis can be calculated using flow cytometry analysis (described above). The percentage of phagocytosis equals the number of macrophages that have engulfed Alexa Fluor 594-zymosan or A. fumigatus-dsRed. Quantification of the extent of phagocytosis can be representative of the mean fluorescence intensity (MFI) of the engulfed Alexa Fluor 594-zymosan or A. fumigatus-dsRed.

Class III PI(3)K activity assay. LAPosomes can be purified as known in the art. mVPS34 can be immunoprecipitated and incubated with phosphatidylinositol (PI). The quenched PI(3)K reactions can then be subjected to a Class III PI(3)K Activity Assay (Echelon Biosciences), a competitive ELISA in which the signal is inversely proportional to the amount of PtdIns(3)P produced. Reaction products can be diluted and added to the PtdIns(3)P-coated microplate, for competitive binding to a PtdIns(3)P detector protein. The amount of PtdIns(3)P detector protein bound to the plate can be determined through colorimetric detection. Data (mean±s.d.) represent three independent experiments in which technical triplicates per sample were acquired using a SpectraMax Microplate Reader (Molecular Devices).

Immunofluorescence. Cells grown and stimulated in chamber slides can be fixed with 4% formaldehyde for 20 min at 4 C. Following fixation, cells can be blocked and permeabilized in block buffer (1% BSA, 0.1% Triton X-100 in PBS) for 1 h at room temperature. Cells can be incubated overnight at 4 C with primary antibody diluted 1/200 in block buffer. Cells can be washed extensively in TBS-Tween (Tris-buffered saline containing 0.05% Tween-20) and incubated with Alexa Fluor-conjugated secondary antibodies (Invitrogen). Images can be analysed using an Olympus BX51 FL Microscope and Slidebook software. Alexa Fluor 647-LAMP1 (clone eBiolD4B, catalogue no. 51-1071) antibody was from eBioscience. Anti-oxLDL (catalogue no. bs-1698R) antibody can be from Bioss Antibodies, and anti-PtdIns(3)P (catalogue no. Z-P003) antibody can be from Echelon Biosciences. Anti-LC3B (catalogue no. ab48394) antibody can be from abCam. Anti-Beclin1 (clone D40C5, catalogue no. 3495), anti-UVRAG (clone D2Q1Z, catalogue no. 13115), anti-VPS34 (clone D9A5, catalogue no. 4263), anti-Rubicon (clone D9F7, catalogue no. 8465), anti-p-p40PHOX (catalogue no. 4311) and anti-ATG14 (catalogue no. 5504) antibodies can be from Cell Signaling. Anti-ATG7 (catalogue no. A2856) antibody can be from Sigma-Aldrich. p22PHOX (clone C17, catalogue no. 11712) antibody can be from Santa Cruz Biotechnology. All primary antibodies can be used at a 1:100 dilution. All secondary antibodies can be used at 1:400. Representative images from reproducible independent experiments can be shown.

1. A method for treating cancer in a subject, said method comprising
administering an effective amount of a LC3-associated phagocytosis (LAP) inhibitor to the subject.

2. The method of claim 1, wherein the LAP inhibitor reduces the expression or activity of at least one gene selected from the group consisting of: Beclin1, VPS34, UVRAG, ATG5, ATG12, ATG16L, ATG7, ATG3, ATG4, LC3A, LC3B, GATE16, GABARAP, Rubicon, and NOX2 is reduced.

3. The method of claim 1, wherein the LAP inhibitor reduces the expression or activity of ATG5.

4. The method of claim 1, wherein said cancer is a cancer attacked by T-cell mediated immunity.

5. The method of claim 1, wherein said cancer is acute myeloid gliomas, histiocytic lymphoma, non-Hodgkin's lymphoma, thyroid cancer, papillary thyroid carcinoma, head and neck cancer, liver cancer, colorectal cancer, pancreatic cancer, breast cancer, ovarian cancer, lung cancer, carcinoma, lymphoma, blastoma, medulloblastoma, retinoblastoma, sarcoma, liposarcoma, synovial cell sarcoma, neuroendocrine tumors, carcinoid tumors, gastrinoma, islet cell cancer, mesothelioma, schwannoma, acoustic neuroma, meningioma, adenocarcinoma, leukemia or lymphoid malignancies, squamous cell cancer, epithelial squamous cell cancer, small-cell lung cancer (SCLC), non-small cell lung cancer (NSCLC), adenocarcinoma of the lung, squamous carcinoma of the lung, cancer of the peritoneum, hepatocellular cancer, gastric or stomach cancer, gastrointestinal cancer, glioblastoma, cervical cancer, bladder cancer, hepatoma, metastatic breast cancer, colon cancer, rectal cancer, endometrial or uterine carcinoma, salivary gland carcinoma, kidney or renal cancer, prostate cancer, vulval cancer, hepatic carcinoma, anal carcinoma, penile carcinoma, Merkel cell cancer, mycoses fungoids, testicular cancer, esophageal cancer, tumors of the biliary tract, or hematological malignancies.

6. The method of claim 1, wherein said effective amount of a LAP inhibitor is administered by inhalation, intranasally, orally, intravenously, subcutaneously, or intramuscularly.

7. The method of claim 1, wherein the effective amount of a LAP inhibitor is administered to the subject in need thereof, in the form of a solution, dispersion, suspension, powder, capsule, tablet, pill, time release capsule, time release tablet, and time release pill.

8. The method of claim 1, further comprising treating the subject with radiation before, during, or after the administration of the LAP inhibitor

9. The method of claim 1, further comprising administering a chemotherapeutic agent to said subject.

10. The method of claim 1, wherein said cancer comprises at least one tumor, and wherein the number of said tumors is reduced following administration of the LAP inhibitor compared to the number of tumors prior to administration of the LAP inhibitor.

11. The method of claim 1, wherein said cancer comprises a tumor, wherein the size of said tumor is reduced following administration of the LAP inhibitor compared to the size of the tumor prior to administration of the LAP inhibitor.

12. The method of claim 10, wherein said tumor is a melanoma tumor or a Lewis cell carcinoma.

13. The method of claim 1, wherein administration of the LAP inhibitor reduces the spread or delays the progression of said cancer.

14. The method of claim 1, wherein administration of the LAP inhibitor increases the Th1 response in the subject.

15. The method of claim 1, wherein expression of IFNγ and/or TNFα is increased following administration of the LAP inhibitor.

16. The method of claim 1, wherein the M1 macrophage level is increased following administration of the LAP inhibitor.

17. The method of claim 1 wherein tumor metastasis is reduced following administration of the LAP inhibitor.

18. The method of claim 1, wherein said LAP inhibitor reduces scavenging of reactive oxygen species.

19. The method of claim 1, wherein said LAP inhibitor is DPI.

20. The method of claim 1, wherein said LAP inhibitor is apocynin.

21. The method of claim 20, wherein said tumor metastasis is pulmonary metastasis.

22. The method of claim 1, wherein said LAP inhibitor reduces scavenging of reactive oxygen species.

23. The method of claim 22, wherein said LAP inhibitor is DPI.

24. The method of claim 1, wherein said LAP inhibitor is apocynin.
